(19) United States
(12) Patent Application Publication
(10) Pub. No.: US 2024/0207486 A1
(43) Pub. Date: Jun. 27, 2024

(54) IMPLANTABLE CELL ENCAPSULATION SYSTEMS

(71) Applicant: CORNELL UNIVERSITY, Ithaca, NY (US)

(72) Inventors: Minglin MA, Ithaca, NY (US); Longhai WANG, Ithaca, NY (US); Alexander ERNST, Ithaca, NY (US)

(21) Appl. No.: 18/555,184

(22) PCT Filed: Apr. 14, 2022
(86) PCT No.: PCT/US2022/024800

§ 371 Date: Oct. 12, 2023

Related U.S. Application Data
(60) Provisional application No. 63/174,739, filed on Apr. 14, 2021.

(51) Int. Cl. A61L 27/38; A61L 27/16; A61L 27/18; A61L 27/20; A61L 27/34; A61L 27/52

(52) U.S. Cl. CPC A61L 27/3804; A61L 27/16; A61L 27/18; A61L 27/20; A61L 27/34; A61L 27/3834; A61L 27/52

(57) ABSTRACT

Disclosure herein are an implantable cell containing device, its subcomponent scaffold, methods of making the same and their methods of use. The implantable cell containing device includes a scaffold and a cell-containing hydrogel encapsulating the scaffold. The scaffold has a tracheal-like internal system of continuous air-filled, hydrophobic micro-channels that traverse the scaffold's dimensions and a hydrophilic external surface. The implantable cell containing device, when implanted in a subject, can be used for delivering a therapeutic agent to a subject in need thereof for the treatment of various conditions and diseases.

This application claims the priority benefit of U.S. Provisional Patent Application Ser. No. 63/174,739, filed Apr. 14, 2021, which is hereby incorporated by reference in its entirety.

This invention was made with government support under grant numbers 1R01DK105967-01A1 awarded by the National Institutes of Health and DGE-1650441 awarded by the National Science Foundation. The government has certain rights in the invention.

### FIELD OF THE INVENTION

The present invention relates to implantable cell encapsulation systems, and scaffold sub-components thereof, as well as methods of manufacturing the scaffolds and implantable cell encapsulation systems, and methods of using the same.

### BACKGROUND OF THE INVENTION

Cell-based therapies are attractive treatments for a variety of diseases, such as diabetes (Shapiro et al., “Clinical Pancreatic Islet Transplantation,” Nat. Rev. Endocrinol. 13:268-277 (2017); Farina et al., “Cell Encapsulation: Overcoming Barriers in Cell Transplantation in Diabetes and Beyond,” Adv. Drug Deliv. Rev. 139:92-115 (2019)), liver diseases (Yu et al., “Cell Therapies for Liver Diseases,” Liver Transplantation 18:9-21 (2012)), and hemophilia (Ohmori et al., “New Approaches to Gene and Cell Therapy for Hemophilia,” J. Thromb. Haemost. 13 Suppl. 1:S133-142 (2015); Roth et al., “Nonviral Transfer of the Gene Encoding Coagulation Factor VIII in Patients with Severe Hemophilia A,” New Engl. J. Med. 344:1735-1742 (2001)). In particular, the delivery of islets (or stem cell-derived β-cells) represents a promising therapy for type 1 diabetes (T1D). Islet transplantation via the injection of isolated islets into the liver portal vein or onto the omentum has shown the potential to normalize glycemic control without exogenous insulin in clinical trials, but life-long recipient immunosuppression is required with this procedure (Shapiro et al., “International Trial of the Edmonton Protocol for Islet Transplantation,” New England Journal of Medicine 355:1318-1330 (2006); Baidal et al., “Bioengineering of an Intraabdominal Endocrine Pancreas,” New England Journal of Medicine 376:1887-1889 (2017)).

Cell encapsulation technology offers to protect cells from immune rejection by isolating them from the host using an artificial, semipermeable material, thereby overcoming the need for immunosuppressive agents (Farina et al., “Cell Encapsulation: Overcoming Barriers in Cell Transplantation in Diabetes and Beyond,” Adv. Drug Deliv. Rev. 139:92-115 (2019); Desai et al., “Advances in Islet Encapsulation Technologies,” Nature Reviews Drug Discovery 16:338-350 (2017)). Unlike traditional organ transplantations (e.g., pancreas transplantation), wherein the host circulatory system is connected to the transplanted organ via surgical vascular anastomosis (White et al., “Pancreas Transplantation,” Lancet 373:1808-1817 (2009)), most islet encapsulation devices (i.e., bioartificial pancreas) remain isolated from the host's bloodstream after transplantation. Therefore, encapsulated cells are entirely dependent on O₂ and other nutrients by passive diffusion from the surrounding blood vessels at the exterior of the device (Popel, “Theory of Oxygen-Transport to Tissue,” Critical Reviews in Biomedical Engineering 17:257-321 (1989)). It is well documented that O₂ is more severely limited than other nutrients because of the relative scarcity of extravascular O₂ in vivo (Colton C K, “Oxygen Supply to Encapsulated Therapeutic Cells,” Advanced Drug Delivery Reviews 67-68:93-110 (2014)). In the intraperitoneal site, the O₂ tension (pO₂) is approximately 40 mmHg, and in the subcutaneous site, likely lower (Barkai et al., “Survival of Encapsulated Islets: More than a Membrane Story,” World J. Transplant. 6:69-90 (2016)).

A thoroughly investigated approach to improve graft oxygenation is to supply exogeneous O₂ in situ. The βAir device (Beta-02), for example, supports injections of high concentration O₂ into a gas-permeable chamber adjacent to hydrogel-encapsulated cells (Ludwig et al., “Favorable Outcome of Experimental Islet Xenotransplantation Without Immunosuppression in a Nonhuman Primate Model of Diabetes,” Proc. Natl. Acad. Sci. USA 114:11745-11750 (2017); Evron et al., “Long-Term Viability and Function of Transplanted Islets Macroencapsulated at High Density are Achieved by Enhanced Oxygen Supply,” Sci. Rep. 8:6508 (2018)). Another strategy is the local production of O₂ using chemical reactions (Pedraza et al., “Preventing Hypoxia-Induced Cell Death in Beta Cells and Islets via Hydrolytically Activated, Oxygen-Generating Biomaterials,” Proc. Natl. Acad. Sci. USA 109:4245-4250 (2012); Wang et al., “An Inverse-Breathing Encapsulation System for Cell Delivery,” Science Advances 7:eabd5835 (2021)) or electrolysis (CA Patent Application No. 2924681 to Tempelman et al.; Wu et al., “In Situ Electrochemical Oxygen Generation with an Immunoisolation Device,” Annals of the New York Academy of Sciences 875:105-125 (1999)). Though these strategies have all demonstrated the benefit of adequate O₂ supply to encapsulated cells, remaining limitations include increased device complexity and the requirement of patient compliance to maintain O₂ provision. O₂ transport in hydrogels is invariably dependent on its permeability, the product of the solubility and diffusivity coefficients, both of which are low in aqueous media such as hydrogels and tissue. An alternative, possibly simpler or complementary approach is thus to improve the O₂ permeability of the encapsulating material.

In the absence of supplemental O₂ provision, theoretical analyses suggest that islets should be within a few hundred micrometers from the blood stream in surrounding tissue to avoid hypoxia (Dulong et al., “A Theoretical Study of Oxygen Transfer Including Cell Necrosis for the Design of a Bioartificial Pancreas,” Biotechnol. Bioeng. 96:990-998 (2007); Iwata et al., “Design of Bioartificial Pancreases From the Standpoint of Oxygen Supply,” Artif Organs 42:E168-E185 (2018)). Based on this design principle, the cell module of an encapsulation system should be exceedingly thin to support favorable oxygenation (Barkai et al., “Survival of Encapsulated Islets: More than a Membrane Story,” World J. Transplant. 6:69-90 (2016); Papas et al., “Oxygenation Strategies for Encapsulated Islet and Beta Cell Transplants,” Adv. Drug Deliv. Rev. 139:139-156 (2019)). For spherical microcapsules which are endowed with a high surface area to volume ratio, a diameter of ˜1000 μm is widely used (Colton C K, “Oxygen Supply to Encapsulated Therapeutic Cells,” Advanced Drug Delivery Reviews 67-68:93-110 (2014); Liu et al., “Zwitterionically Modified Alginates Mitigate Cellular Overgrowth for Cell Encapsulation,” Nat. Commun. 10:5262 (2019); Bochenek et al., “Alginate Encapsulation as Long-Term Immune Protection of Allogeneic Pancreatic Islet Cells Transplanted into the Omental Bursa of Macaques,” Nat. Biomed. Eng. 2:810-821 (2018)). Similarly, cylindrical cell-laden hydrogel fibers are commonly designed from 350-1000 μm in diameter (Onoe et al., “Metre-Long Cell-Laden Microfibres Exhibit Tissue Morphologies and Functions,” Nature Materials 12:584-590 (2013); Watanabe et al., “Millimeter-Thick Xenoislet-Laden Fibers as Retrievable Transplants Mitigate Foreign Body Reactions for Long-Term Glycemic Control in Diabetic Mice,” Biomaterials 255:120162 (2020); An et al., “Developing Robust, Hydrogel-Based, Nanofiber-Enabled Encapsulation Devices (NEEDs) for Cell Therapies,” Biomaterials 37:40-48 (2015)), and planar slabs (the geometry endowed with the lowest surface area to volume ratio), typically from 250-600 μm in thickness (Desai et al., “Advances in Islet Encapsulation Technologies,” Nature Reviews Drug Discovery 16:338-350 (2017); Ludwig et al., “Favorable Outcome of Experimental Islet Xenotransplantation Without Immunosuppression in a Nonhuman Primate Model of Diabetes,” Proc. Natl. Acad. Sci. USA 114:11745-11750 (2017); Evron et al., “Long-Term Viability and Function of Transplanted Islets Macroencapsulated at High Density are Achieved by Enhanced Oxygen Supply,” Sci. Rep. 8:6508 (2018)).

Clinical islet transplantations require approximately 500 k islet equivalent (IEQ) of human islets (5-10 k IEQ per kg body weight) to reverse diabetes (Shapiro et al., “Clinical Pancreatic Islet Transplantation,” Nat. Rev. Endocrinol. 13:268-277 (2017)), and cellular treatments for liver diseases and hemophilia require similar cell volumes (Roth et al., “Nonviral Transfer of the Gene Encoding Coagulation Factor VIII in Patients with Severe Hemophilia A,” New England Journal of Medicine 344:1735-1742 (2001)). Because O₂ diffusion limitations restrict hydrogel thickness, devices can only be scaled along one or two spatial dimensions to accommodate this requisite cell payload, and thus an unreasonably large estimated device length, surface area, or number is required. It is estimated that meters of a cylindrical fiber, hundreds of square centimeters of a planar slab, or ˜100,000 microcapsules are needed to deliver a curative islet dose. Increasing the system's O₂ permeability would allow devices to be scaled in three dimensions, facilitating the design of reasonable and surgically convenient device geometries (i.e., shorter in length, smaller in surface area, or lower in number).

The present invention is directed to overcoming these and other deficiencies in the art.

### SUMMARY OF THE INVENTION

A first aspect of the disclosure relates to an implantable cell containing device. This implantable cell containing device includes a scaffold, and a cell-containing hydrogel encapsulating the scaffold. The scaffold has a tracheal-like internal system of continuous air-filled, hydrophobic micro-channels that traverse the scaffold's dimensions and a hydrophilic external surface layer.

A second aspect of the disclosure relates to a scaffold for use in a cell encapsulation system. The scaffold has a three-dimensional structure including a network of hydrophobic microchannels throughout the three-dimensional structure; and a hydrophilic external surface of the three-dimensional structure.

A third aspect of the disclosure relates to a method of preparing a scaffold for a cell encapsulation system. The method includes the steps of: providing a mold; introducing a polymer solution into the mold and allowing the polymer to solidify and form a three-dimensional structure comprising a network of hydrophobic microchannels throughout the three-dimensional structure; and removing the mold to release the three-dimensional structure, thereby forming the scaffold.

A fourth aspect of the disclosure relates to a method of forming a cell encapsulation system suitable for implant. The method includes the steps of: providing a hydrogel precursor solution comprising one or more cells suspended in the hydrogel precursor solution; and combining the hydrogel precursor solution with a scaffold according to the second aspect in a contained state, and allowing the hydrogel precursors to cross-link to form the hydrogel with the scaffold and the one or more cells embedded within the hydrogel.

A fifth aspect of the disclosure relates to a method of delivering a therapeutic agent to a subject in need thereof. This method involves implanting the implantable cell containing device as described herein.

A sixth aspect of the disclosure relates to a method of treating diabetes in a subject. This method involves implanting the implantable cell containing device as described herein into the subject having diabetes. One particular embodiment of this aspect involves the encapsulation of islet cells in the implantable cell containing device, where the islet cells produce insulin, glucagon, or a combination thereof.

A seventh aspect of the disclosure relates to a method of treating a bleeding disorder in a subject. This method involves implanting the implantable cell containing device as described herein into the subject having a bleeding disorder. One particular embodiment of this aspect involves the encapsulation of cells in the implantable cell containing device, where the cells produce and release one or more blood clotting factors.

An eighth aspect of the disclosure relates to a method of treating a lysosomal storage disease in a subject. This method involves implanting the implantable cell containing device as described herein into the subject having the lysosomal storage disease. One particular embodiment of this aspect involves the encapsulation of cells in the implantable cell containing device, where the cells produce and release one or more enzymes that are deficient in the subject and thereby treat the lysosomal storage disease.

A ninth aspect of the disclosure relates to a method of treating a neurological disorder in a subject. This method involves implanting the implantable cell containing device of as described herein into the subject having the neurological disorder. One particular embodiment of this aspect involves the encapsulation of cells in the implantable cell containing device, where the cells produce and release one or more molecules that are effective to treat the neurological disorder.

A tenth aspect of the disclosure relates to a method of treating a cancer in a subject. This method involves implanting the implantable cell containing device as described herein into the subject having cancer. One particular embodiment of this aspect involves the encapsulation of cells in the implantable cell containing device, where the cells produce and release one or more molecules that are effective to treat the cancer.

An eleventh aspect of the disclosure relates to a method of treating a chronic eye disease in a subject. This method involves implanting the implantable cell containing device as described herein into the subject having a chronic eye disease. One particular embodiment of this aspect involves the encapsulation of cells in the implantable cell containing device, where the cells produce and release one or more molecules that are effective to treat the chronic eye disease.

A twelfth aspect of the disclosure relates to a method of treating kidney failure in a subject. This method involves implanting the implantable cell containing device as described herein into the subject having a kidney failure. One particular embodiment of this aspect involves the encapsulation of cells in the implantable cell containing device, where the cells produce and release one or more molecules that are effective to treat the kidney failure.

A thirteenth aspect of the disclosure relates to a method of treating a chronic pain in a subject. This method involves implanting the implantable cell containing device as described herein into the subject having a chronic pain. One particular embodiment of this aspect involves the encapsulation of cells in the implantable cell containing device, where the cells produce and release one or more molecules that are effective to treat the chronic pain.

Inadequate oxygenation is a major challenge in cell encapsulation, a therapy which holds potential to treat many diseases including type I diabetes. In such systems, cellular oxygen (O₂) delivery is limited to slow passive diffusion from transplantation sites through the poorly O₂-soluble encapsulating matrix, usually a hydrogel. This constrains the maximum permitted distance between the encapsulated cells and host site to within a few hundred micrometers to ensure cellular function.

The physiology of insects presents a creative solution to rapid O₂ distribution across multi-millimeter scales. Instead of using circulatory blood for tissue oxygenation as in vertebrates (Pittman, “Regulation of Tissue Oxygenation,” Colloquium Series on Integrated Systems Physiology: from Molecule to Function, Morgan & Claypool Life Sciences (2011), which is hereby incorporated by reference in its entirety), insects transport gaseous oxygen via a gas-filled channel network, known as the tracheal system, which is distributed throughout their bodies. This system is also present in some aquatic insects without spiracles (Klowden, Physiological Systems in Insects, Third Edition, Chapter 7: “Circulatory Systems,” pp. 357-401, and Chapter 9: “Respiratory Systems,” pp. 433-461, Elsevier (2013), which is hereby incorporated by reference in its entirety). Inspired by this clever mechanism of rapid gas-phase O₂ distribution, the present invention includes the design of an air-filled scaffold for islet encapsulation, which was pursued to overcome the thickness limitation for O₂ diffusion and thereby termed Speedy Oxygenation Network for Islet Constructs (or SONIC). See FIGS. 1A-K.

The bio-inspiration was highlighted by analyzing the tracheal anatomy of a larva of the mealworm beetle (Tenebrio molitor) (FIG. 1A), which guided the design of the SONIC scaffold. High-resolution X-ray computer tomography (Nano-CT) scanning was used to visualize the tracheal network in the mealworm, showing its thorough distribution throughout the body in a ladder-like geometry (Ras et al., “The Tracheal System in Post-Embryonic Development of Holometabolous Insects: A Case Study Using the Mealworm Beetle,” J. Anat. 232:997-1015 (2018); Iwan et al., “The Last Breath: A CT-Based Method for Investigating the Tracheal System in Hexapoda,” Arthropod Structure & Development 44:218-227 (2015), which are hereby incorporated by reference in their entirety) (FIG. 1B). It was evident from this pattern that O₂ was delivered to insect tissue through the air channels in the tracheae (FIG. 1C). Importantly, the O₂ diffusion coefficient in the gaseous phase was around 10⁴ times higher than that in water or biological tissue (Klowden, Physiological Systems in Insects, Third Edition, Chapter 9: “Respiratory Systems,” pp. 433-461, Elsevier (2013); Couzin et al., “Effective Leadership and Decision-Making in Animal Groups on the Move,” Nature 433:513-516 (2005), which are hereby incorporated by reference in their entirety). Thus, the tracheal network leverages rapid gas-phase O₂ transport, allowing insects to efficiently distribute O₂ throughout their bodies (Polet, “The Biggest Bugs: An Investigation into the Factors Controlling the Maximum Size of Insects,” Eureka 2:43-46 (2011), which is hereby incorporated by reference in its entirety). This innovative O₂ transport mechanism allows some insects to grow to incredibly large sizes (Harrison et al., “Atmospheric Oxygen Level and the Evolution of Insect Body Size,” Proceedings of the Royal Society B: Biological Sciences 277:1937-1946 (2010), which is hereby incorporated by reference in its entirety), such as the Goliath beetle larvae reaching the size of a mouse, the Dynastes hercules larvae reaching 147 mm long with weight of 144 g, and the Megasoma larvae reaching a maximum dorsal width of 225 mm (Acorn J. “The World's Biggest Bug is a Grub,” American Entomologist 52:270-272 (2006), which is hereby incorporated by reference in its entirety).

The incorporation of the SONIC scaffold in a hydrogel-based cell encapsulation system described in the present application intentionally recapitulated the efficiency of rapid O₂ transport of the insect tracheal network, oxygenating deeply encapsulated cells within the thick devices (FIG. 1D). The biomimetic scaffold features internal continuous air channels endowed with 10,000-fold higher O₂ diffusivity than hydrogels. The scaffold was incorporated into a bulk hydrogel containing cells, which facilitated rapid O₂ transport through the whole system to cells several millimeters away from the device-host boundary. A computational model, validated by in vitro analysis, predicted that cells and islets maintain high viability even in a thick (6.6 mm) device. Additionally, it was demonstrated that the SONIC device can maintain high cell viability and robust function of rat islets in immunocompetent diabetic mice for over 6 months. The biomimetic SONIC cell delivery system solved the problem of slow and non-penetrating O₂ transport in thick bulk hydrogels.

### BRIEF DESCRIPTION OF THE DRAWINGS

FIGS. 1A-K show the design and fabrication of the biomimetic SONIC scaffold. FIG. 1A is a digital image of a larva of the mealworm beetle (Tenebrio molitor). FIG. 1B is a 3D reconstruction of Nano-CT images of the tail of a mealworm (left) and the segmented gas phase tracheal system (right) inside the body. FIG. 1C is a schematic illustrating the tracheal system in a mealworm and O₂ delivery to the surrounding cellular tissue through the tracheae. FIG. 1D is a schematic illustrating O₂ delivery from the transplantation site into the cell encapsulation system through a tracheal ladder network-like SONIC scaffold. FIG. 1E shows the chemical structure of the fluoropolymer PVDF-HFP. FIG. 1F shows fabrication of the ladder-like SONIC scaffold. FIG. 1G is a digital image of the SONIC scaffold. FIG. 1H shows schematics representing the macro- and microarchitecture structure of the SONIC scaffold. FIG. 1I is a nano-CT image of the porous scaffold (the asterisks indicate the pore regions). FIG. 1J is a 3D reconstruction of Nano-CT images of a selected region (26×34.84×5.36 μm) inside the SONIC scaffold showing the bicontinuous microstructure. FIG. 1K shows skeletal networks for the polymeric (upper/middle panels) and the porous (lower/middle panels) regions of the SONIC scaffold.

FIGS. 2A-M show the fabrication and characterizations of the SONIC device. FIG. 2A is a schematic illustration of the device fabrication (side view). FIGS. 2B-C show false-colored SEM images of the SONIC scaffold (FIG. 2B) and polydopamine-coated SONIC scaffold (FIG. 2C). One representative of 3 independent experiments is shown. FIGS. 2D-E show digital images of water droplets (colored with food dye) and contact angle goniometer-captured images of a water droplet on a rectangular prism SONIC scaffold before (FIG. 2D) and after (FIG. 2E) polydopamine modification. FIG. 2F shows a false-colored SEM image of the polydopamine-coated SONIC scaffold with deposited CaSO₄ crystals. One representative of 3 independent experiments is shown. FIG. 2G is a stereo microscope image of the SONIC device (top view). FIG. 2H shows SEM/EDS elemental mapping of F, N, and S on a polydopamine-coated SONIC scaffold with deposited CaSO₄ crystals. The arrows indicate the lack of polydopamine at a coating crack location. One representative of 3 independent experiments is shown. FIG. 2I shows SEM image of the polydopamine-coated SONIC scaffold and the corresponding element N distribution profile across a polydopamine coating crack. One representative of 3 independent experiments is shown. FIG. 2J is a SEM image of the cross-sectional polydopamine-modified SONIC scaffold, showing no polydopamine inside the scaffold. One representative of 3 independent experiments is shown. FIG. 2K shows an H&E staining slice of an islet encapsulation device showing the polydopamine located at the interface between the SONIC scaffold and alginate hydrogel. One representative of 10 replicates is shown. FIG. 2L shows images captured during the perfusion test using a cylindrical SONIC scaffold. A pump-connected needle was inserted into one end of a cylindrical SONIC scaffold, and the other end of SONIC scaffold was immersed into a vial containing water phase (top, colored with green food dye) and chloroform phase (bottom, colored by Nile Red dye). FIG. 2M is a schematic representing the distribution of water and chloroform during the perfusion test.

FIGS. 3A-I show characterizations of rapid O₂ transport through the SONIC scaffold. FIG. 3A shows a digital image (left) and schematic (right) showing the sample in container for the EPR test. FIG. 3B shows the chemical structure of the EPR spin probe OX063-d24. FIG. 3C shows a calibration curve of the OX063-d24 relaxation rate versus pO₂. FIG. 3D is a graph showing average pO₂ of gelatin in the container with a PLA control insert versus time. The initial gray period indicates the deoxygenation of the system. FIG. 3E shows pO₂ distributions on tangential plane and transverse plane of sample with the PLA control insert at different time points (indicated by the arrows in FIG. 3D). FIG. 3F is a graph showing an average pO₂ of gelatin in the container with a SONIC scaffold versus time showing much faster equilibration. FIG. 3G shows pO₂ distributions on a tangential plane and a transverse plane of sample with the SONIC scaffold at different time points (indicated by the arrows in FIG. 3F). FIG. 3H shows simulation data showing spatial pO₂ profiles over time in system with the PLA control insert and SONIC scaffold. FIG. 3I is a graph showing simulated average pO₂ of gelatin in the container versus time.

FIGS. 4A-L show that the SONIC scaffold improves cell survival under hypoxic conditions. FIG. 4A shows simulation-predicted pO₂ distributions of INS-1 cells encapsulated control device in three tangential cross sections (labeled A-A, B-B, and C-C) and a transverse cross section (labeled D-D, a corresponding cₒ_{₂} distribution labeled as D′-D′). FIG. 4B shows quantitative pO₂ and cₒ_{₂} distributions along a radial line in the transverse cross section of the control device showing rapid O₂ dropping from 40 mmHg (0.05 mM) at the surface to −3 mmHg (0.004 mM) at the center. FIG. 4C shows simulation-predicted pO₂ distributions of INS-1 cells encapsulated SONIC device in three tangential cross sections and a transverse cross section. FIG. 4D shows quantitative pO₂ and cₒ_{₂} distributions along a radial line in the transverse cross section of the SONIC device showing high pO₂ over 35 mmHg in the whole device, corresponding with high cₒ_{₂} over 0.044 mM in the cell/hydrogel phase and a substantially higher cₒ_{₂} of ˜-1.85 mM in the SONIC scaffold due to the preferential partitioning of O₂ into the gas-containing SONIC scaffold. FIGS. 4E-F show microscope images of live/dead staining of INS-1 cells encapsulated in the control device (FIG. 4E) and the SONIC device (FIG. 4F). One representative of 2 independent experiments is shown. FIG. 4G shows simulation-predicted pO₂ distributions of rat islets (size-distributed) in a control device in three tangential cross sections (labeled A-A, B-B, and C-C) and two transverse cross sections (labeled D-D and E-E) showing massive hypoxic regions in the center of the device with necrosis observed in many islets (white regions in the islets represent necrosis). FIG. 4H shows simulation-predicted pO₂ distributions of rat islets (size-distributed) in a SONIC device in three tangential cross sections (labeled A-A, B-B, and C-C) and two transverse cross sections (labeled D-D and E-E) showing well-oxygenated islets in the entire device, with negligible necrosis observed in rare large islets (arrow in A-A cross section). FIGS. 4I-J are graphs showing average pO₂ (FIG. 4I) and fraction of necrosis (FIG. 4J) of the islet populations in control devices, and SONIC devices. Mean±SD, ****p<0.0001 (unpaired two-tailed students t-test). FIGS. 4K-L are graphs showing scatter plots of islet location versus average pO₂ in simulated islets (all 150 μm in diameter) in control devices (FIG. 4K) and SONIC devices (FIG. 4L).

FIGS. 5A-M show that the SONIC device enables 6-month diabetes correction in mice. FIGS. 5A-B show schematic (FIG. 5A) and microscope image (FIG. 5B) of rat islets encapsulated in a SONIC device (˜4 mm in diameter). One representative of 11 replicates is shown. FIG. 5C shows BG measurements of diabetic C57BL6/J mice following IP transplantation of SONIC devices (pink, n=5, retrieved on day 60; red, n=6, one device was retrieved on day 145 after BG rising and the other five were retrieved on day 181), or control devices (black, n=5, two mice were sacrificed on day 96 and day 128 due to poor health, the other three devices were retrieved on day 181); ****p<0.0001 (one-way analysis of covariance (ANCOVA)). FIG. 5D shows IPGTT on day 58; mean±SD; ****p<0.0001 (diabetic mice versus SONIC device-treated mice, diabetic mice versus healthy mice), n.s. (p>0.05; SONIC device-treated mice versus healthy mice). FIG. 5E shows IPGTT on day 180; mean±SD; ****p<0.0001 (diabetic mice versus SONIC device-treated mice, diabetic mice versus healthy mice, control device-treated mice versus SONIC device-treated mice, and control device-treated mice versus healthy mice), n.s. (p>0.05; diabetic mice versus control device-treated mice and SONIC device-treated mice versus healthy mice). Statistical tests in FIGS. 5D and 5E were analyzed via a two-way analysis of variance (ANOVA) followed by Sidak's post hoc p-value adjustment for multiple comparisons. FIG. 5F is a graph showing static GSIS test of devices (n=3) retrieved on day 60; mean±SD, ***p<0.001 (paired two-tailed students t-test). FIG. 5G is an image showing live/dead staining of islets from a retrieved SONIC device on day 60. One representative of 2 replicates is shown. FIGS. 5H-I show H&E and immunohistochemical staining of tangential cross sections of retrieved devices on day 60 showing intact morphology and insulin/glucagon-positive islets in both peripheral regions (FIG. 5H) and central regions (FIG. 5I) of the device. One representative of 3 replicates is shown. FIG. 5J shows H&E staining of the transverse section of a retrieved device on day 181 showing healthy islets in both peripheral regions and central regions of the device. The asterisk indicates the host side of the device-host interface. One representative of 2 replicates is shown. FIG. 5K shows a selected transverse surface plot collected from the simulation device showing well oxygenated islets in both peripheral regions and central regions. FIG. 5L shows H&E staining of a retrieved device on day 181 showing healthy islets in device even with some deposited fibrosis on the device. The asterisk indicates the host side of the device-host interface. One representative of 4 replicates is shown. FIG. 5M shows selected surface plots collected from the simulation of devices with fibrosis on one half of the device (implemented by a no-flux condition on the bottom half). The PLA control device (left) showed a significant lower pO₂ at the blocked bottom side in comparison to the unblocked top side, and a necrotic islet (arrow, left panel) was observed near the blocked face. The SONIC device (right) showed a slighter lower pO₂ at the blocked bottom side in comparison to the unblocked top side, but the islets (arrows) a, right panelt both sides were sufficiently oxygenated.

FIGS. 6A-M show demonstration of the therapeutic potential of a thick SONIC device. FIGS. 6A-B are schematics representing the simulated geometry for a thick control device (FIG. 6A) and thick SONIC device (6.6×6.6×6.6 mm) (FIG. 6B) containing rat islets (size-distributed). Simulation-predicted pO₂ distributions of the control device in three cross sections (labeled A-A, B-B, and C-C) showing a massive hypoxic central region, with necrosis observed even in some small islets (arrows in C-C cross section). However, the islets were well oxygenated throughout the SONIC device with negligible necrosis. FIGS. 6C-D are graphs showing average pO₂ (FIG. 6C) and fraction of necrosis (FIG. 6D) in the islet populations in control devices and SONIC devices at size of 4.2 mm and 6.6 mm, mean±SD. FIGS. 6E-F show Scatter plots of islet location versus average pO₂ of islets (generated as all 150 μm in diameter) in the thick control device (FIG. 6E) and thick SONIC device (FIG. 6F). FIGS. 6G-H show schematic (FIG. 6G) and microscope image (FIG. 6H) of rat islets encapsulated in a thick cubic SONIC device with a side length of ˜6.6 mm. One representative of 5 replicates is shown. FIG. 6I shows BG measurements of diabetic C57BL6/J mice following IP transplantation of the 6.6 mm SONIC devices (pink, n=5, retrieved on day 60; red, n=5, retrieved on day 123). FIG. 6J shows IPGTT on day 58; mean±SD; **p<0.01 (two-way ANOVA). FIG. 6K shows live/dead staining of islets from a retrieved device on day 120. FIGS. 6L-M show H&E (FIG. 6L) and immunohistochemical staining (FIG. 6M) of retrieved devices on day 120 showing morphology-intact and insulin/glucagon-positive islets. One representative of 5 replicates is shown.

FIGS. 7A-F show computational exploration of a SONIC spiral device for delivering a clinically relevant islet dose. FIG. 7A is an annotated schematic of the central section of a hypothetical SONIC spiral device, including the SONIC scaffold arranged in an Archimedean spiral (with the distance between turns fixed at 500 m) and hydrogel-encapsulated human islets. A thickness of 1.2 mm ensures a maximum distance of insulin diffusion of 600 μm; a diameter of 4.75 mm was used for simulations, representing the central section of a device scaled radially to achieve a sufficient encapsulated islet payload. FIG. 7B are schematics showing the SONIC spiral device and scaffold-free control device encapsulating 4%, 6%, 8%, and 10% human islets (volume of islets per volume of device), as tested in the simulations. FIGS. 7C-D are graphs showing simulation predictions of the mean islet population pO₂ (FIG. 7C) and fraction of necrosis (FIG. 7D) of human islets encapsulated at variable densities in the SONIC spiral device and the scaffold-free control device; mean±SD; FIG. 7C: ****p<0.0001 (control device versus SONIC spiral device at all islet densities); FIG. 7D: ***p<0.001 (control device versus SONIC spiral device at 4% islet density), **p<0.01 (control device versus SONIC spiral device at 6%), ****p<0.0001 (control device versus SONIC spiral device at 8% and 10% islet densities). Statistical tests in FIGS. 7C and 7D were analyzed via a two-way ANOVA followed by Sidak's post hoc p-value adjustment for multiple comparisons. FIGS. 7E-F show surface plots of pO₂ gradients (right) at selected cross sections (left; labelled A-A, B-B, and C-C) in the control device (FIG. 7E) and the SONIC spiral device (FIG. 7F) at 8% human islet loading density showing significantly higher pO₂ and negligible necrosis in the SONIC spiral device compared to the control device (white regions in the islets represent necrosis, and yellow arrows in C-C section indicate necrosis even in some small islets).

FIG. 8 is a schematic showing the geometry of the thick cubic empty control (left), SONIC device (middle), and annotated dimensions (right) of simulations featuring size-distributed islets.

FIG. 9 is an annotated schematic depicting the dimensions of a planar device featuring a SONIC scaffold in an Archimedean spiral configuration, which was used for simulations.

FIG. 10A is an image of the 3D-printed PLA mold, and FIG. 10B is an image of a fabricated four-layer thick SONIC scaffold using the mold.

FIG. 11A is an image of toroidal SONIC scaffolds, and FIG. 11B is a microscopy image of a toroidal SONIC device with rat islets embedded in the hydrogel.

FIG. 12 is a schematic showing the geometry of a disk-like SONIC device illustrating the intertwined spiral SONIC scaffold and hydrogel phase (analogous to a rolled hydrogel sheet).

FIG. 13 is a schematic of a unit in the SONIC device illustrating the intertwined SONIC scaffold and hydrogel phase, and individually analogic “microcubes” with surrounding O₂ from the transplantation site.

### DETAILED DESCRIPTION OF THE INVENTION

The present disclosure relates to implantable cell containing devices, methods of producing these devices, and methods of using the same.

One aspect of the disclosure relates to an implantable cell containing device. This implantable cell containing device includes a scaffold and a cell-containing hydrogel encapsulating the scaffold. The scaffold includes a tracheal-like internal system of continuous air-filled, hydrophobic micro-channels that traverse the scaffold's dimensions, and has a hydrophilic external surface.

The scaffold may have any of a variety of constructions such that the bulk of the hydrogel material that surrounds the scaffold, and the cells that are contained in the hydrogel, is not more than about 1000 μm from the external surface of the scaffold, such as about 900 μm from the external surface of the scaffold, about 800 μm from the external surface of the scaffold, about 700 μm from the external surface of the scaffold, or 600 μm from the external surface of the scaffold. In certain embodiments, the bulk of the hydrogel material that surrounds the scaffold, and the cells that are contained in the hydrogel, is not more than about 500 μm from the external surface of the scaffold, preferably not more than about 450 μm, or about 400 μm, or about 350 μm, or about 300 μm, or about 250 μm from the external surface of the scaffold. This helps to ensure that, via diffusion of oxygen from the scaffold into the hydrogel matrix, the cells contained in the hydrogel matrix remain viable.

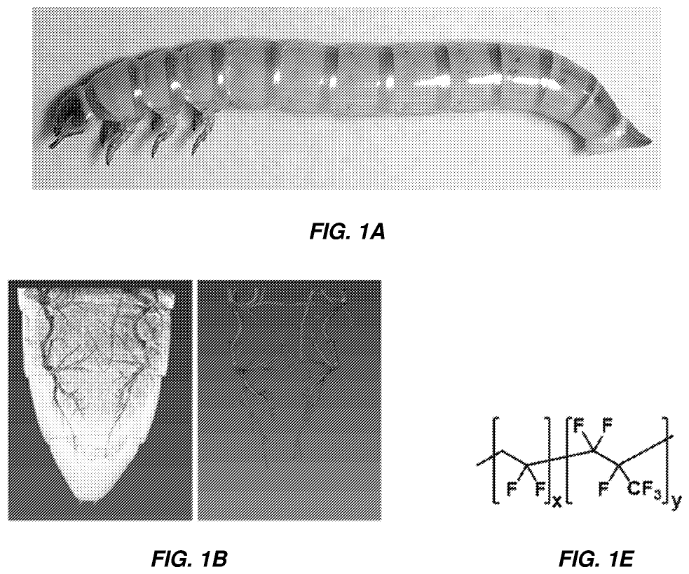

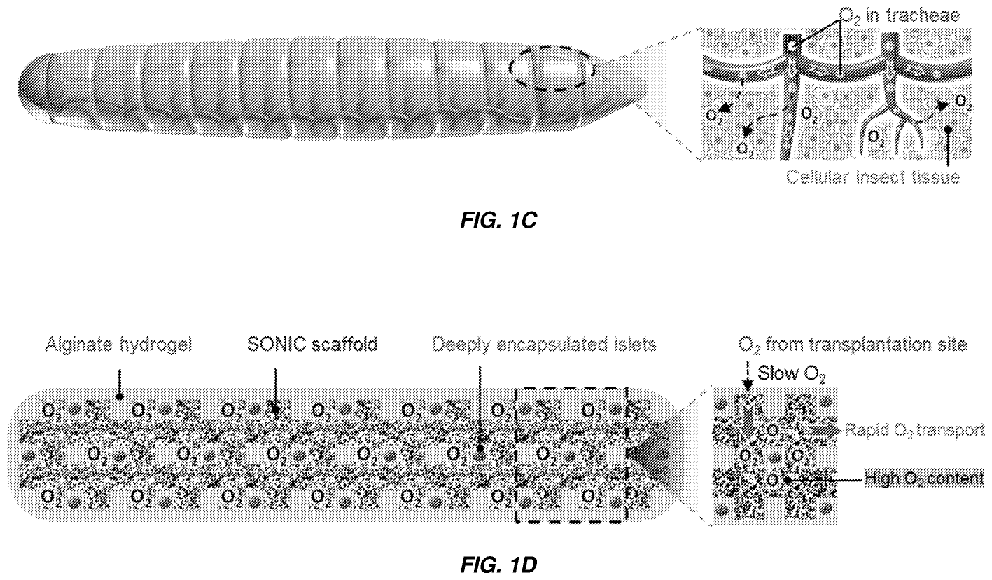

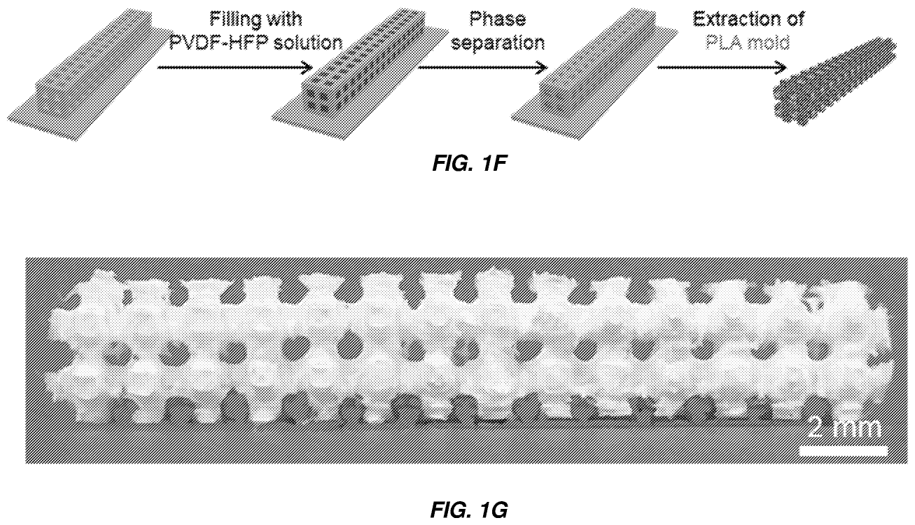

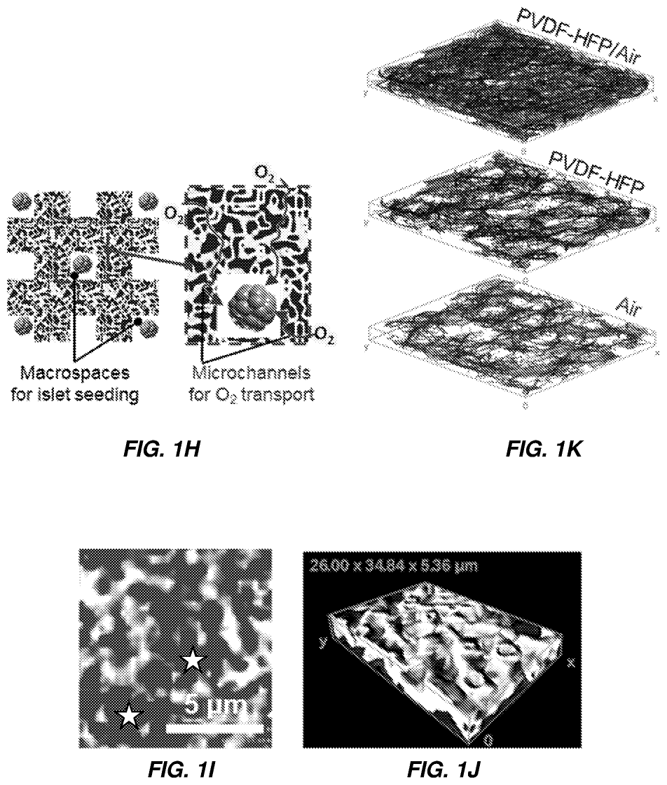

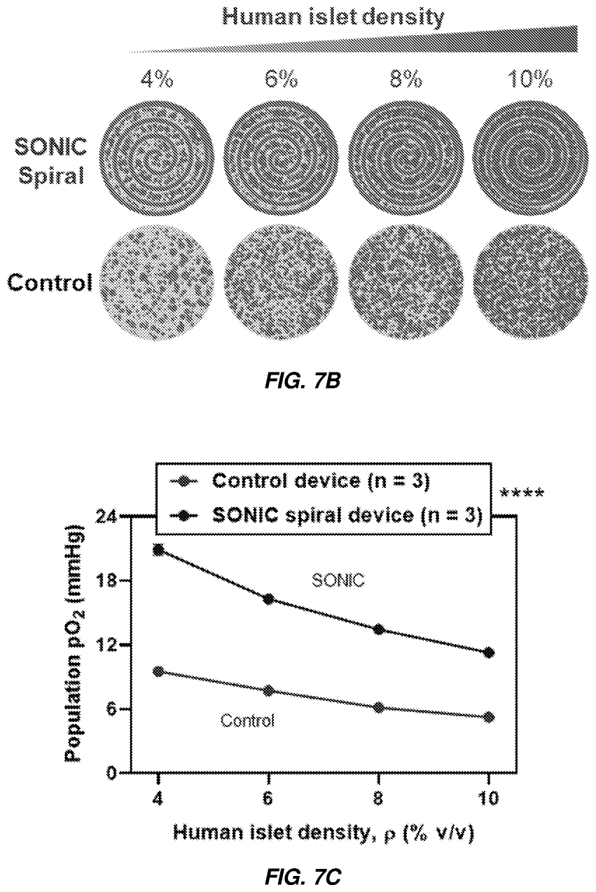

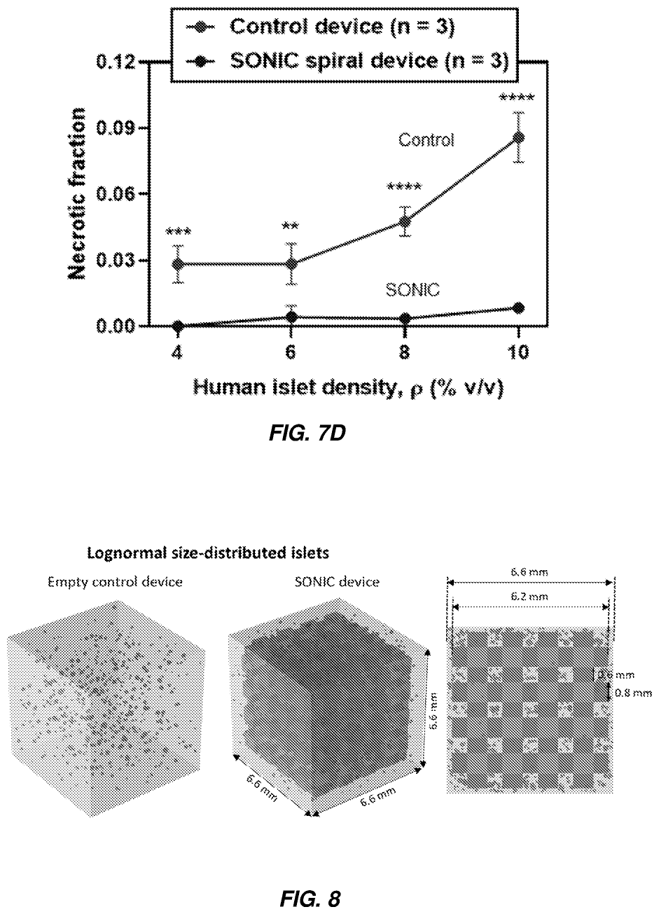

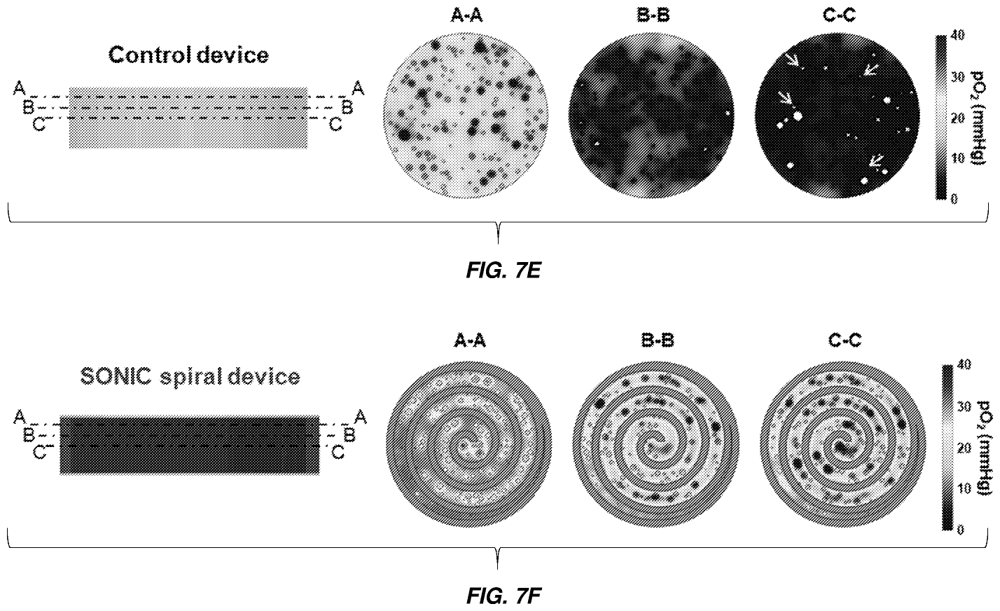

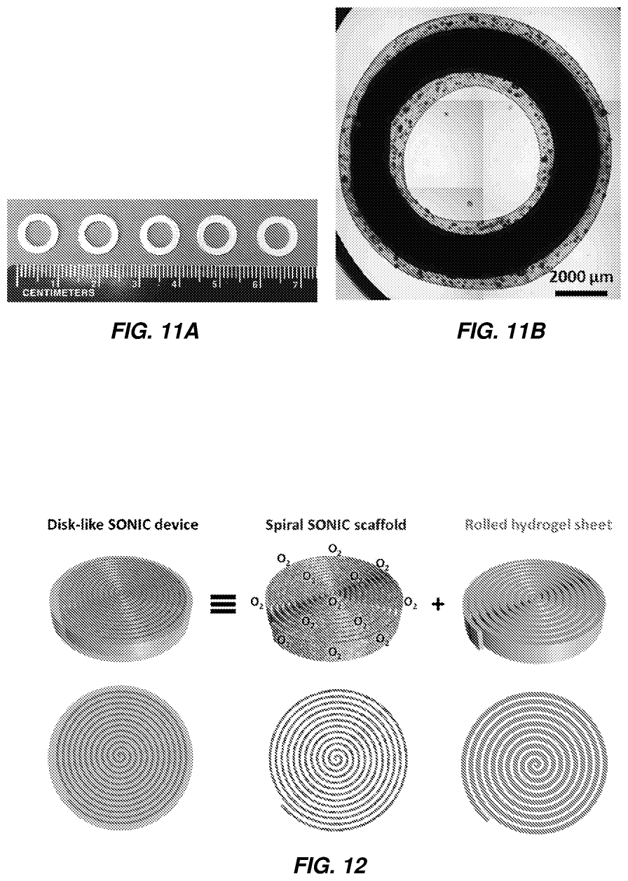

The variety of scaffold constructions are not limited to any particular shape or configuration. Exemplary shapes or configurations are presented in the accompanying examples, and include a ladder-like geometry with varying heights and lengths (see FIGS. 1G, 10B, 13), a spiral geometry (see FIGS. 7A-F, 9, 12), and a toroidal geometry (see FIGS. 11A-B). Other suitable geometries include, without limitation, a planar geometry, a rod-shaped geometry, and a tubular geometry.

The scaffold has an internal microstructure that is bi-continuous and non-wettable (hydrophobic) to provide unobstructed microchannels for O₂ flow similar to the insect tracheae. The scaffold also has an external surface that is rough and wettable (hydrophilic) to allow cell-laden hydrogel precursor to infiltrate. Although the architecture of the channels in the scaffold are largely in the micron range, it is to be understood that sub-micron channels may or may not be present. Thus, as used herein, the term ‘microchannels’ also encompasses channels that would be considered nanochannels due to their dimensions.

According to one embodiment, a hydrophobic polymer material is used to form the scaffold.

In some embodiments, the scaffold comprises a fluorinated polymer material. Suitable fluorinated polymer materials that can be used include, without limitation, poly(vinylidene fluoride-co-hexafluoropropylene) (PVDF-HFP), poly(vinylidene fluoride) (PVDF), polyvinylidene difluoride, polytetrafluoroethylene (PTFE), poly(vinylidene fluoride-co-trifluoroethylene) (P(VDF-TrFE)), poly(vinylidene fluoride-co-tetrafluoroethylene) (P(VDF-TFE)), poly(vinylidene fluoride-co-chlorotrifluoroethylene) (P(VDF-CTFE)), Teflon AF® family: copolymers made from 2,2-bistrifluoromethyl-4,5-difluoro-1,3-dioxole and tetrafluoroethylene, and polychlorotrifluoroethylene (PCTFE).

Other suitable polymer materials that can be used include, without limitation, silicone, PDMS, rubber, nylon, polyurethane, polysulfone, polyacrylonitrile, polyester such as polyethylene terephthalate and polybutester, polyacrylamide, poly(ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(1-lactide-co-ε-caprolactone), and combinations thereof.

In an alternative embodiment, when a non-hydrophobic polymer material is used to form the scaffold, the microchannels of the scaffold are subsequently treated to render the microchannels hydrophobic (non-wettable). In one approach, the microporous scaffold can be chemically modified with fluoroalkysilanes to render the microchannels hydrophobic. Alternatively, the microchannels in the scaffold can be modified to comprise SiO₂ nanoparticles, attapulgite, ZnO nanoparticles or nanorods, and combinations thereof.

In yet another embodiment, the scaffold is formed of a carbon material. Suitable carbon materials that can be used include, without limitation, activated carbon, carbon microbelts, graphite, carbon nanoparticles, carbon soot, carbon nanofibers, graphene, and carbon nanotubes.

In alternative embodiments, a mixture of carbonaceous materials and polymer materials are used to form the scaffold.

For scaffold materials that are hydrophobic in nature, the external surface of the scaffold should be treated to render the external surface hydrophilic. In certain embodiments, a hydrophilic polymer coating is applied to the external surface. Suitable hydrophilic coating materials include, without limitation, polydopamine coatings and silane coatings, such as PEGylated silane coatings.

In an alternative embodiment, the external surface of the scaffold can be treated by exposing the scaffold to plasma (e.g., atmospheric), which will render the otherwise hydrophobic polymer surface hydrophilic.

The inventive scaffolds can be prepared by first preparing or providing a mold, and then introducing a polymer solution into the mold and allowing the polymer to solidify and form a three-dimensional structure comprising a network of microchannels throughout the three-dimensional structure, and then removing the mold to release the three-dimensional structure, thereby forming the scaffold. This is generally illustrated in FIG. 1F with respect to the exemplary materials described in the accompanying Examples. This process is not limited to the exemplary materials.

In certain embodiments, the mold is prepared using a three-dimensional printing technique that forms a patterned mold, which has a pattern that allows for development of the desired three-dimensional structure of the scaffold. The mold is shown to the left side of FIG. 1F. A number of suitable material exist for use in forming the mold, including without limitation, poly(lactic acid) (PLA), polyethylene terephthalate (PETG), acrylonitrile butadiene styrene (ABS), acrylic styrene-acrylonitrile (ASA), nylon, polyvinyl alcohol (PVA), polycarbonate (PC), polypropylene (PP), polystyrene (PS), polyvinyl butyral (PVB), thermoplastic polyurethane (TPU), and co-polyester (CPE).

The step of introducing a polymer solution into the mold and allowing the polymer to solidify and form a three-dimensional structure is carried out by phase separation technique. As demonstrated in the accompanying examples, phase separation is a method for forming the scaffold by precipitation of polymers from a polymer-poor phase and a polymer-rich phase. The advantage of the phase separation process is that it is a relatively simple procedure and requires minimal apparatus (i.e., the mold) and suitable solvent/nonsolvent for a given polymer. Most polymer solution systems have a suitable, corresponding poor solvent (nonsolvent) to create a porous structure using the phase separation technique. In this process, the polymer is dissolved in solution and the phase separation is induced, either thermally or through the addition of a non-solvent to the polymer solution to create a gel. The polymer solution under this condition becomes thermodynamically unstable and tends to separate into two phases. Water is then used to extract the solvent from the gel; the polymer-rich phase then solidifies on reducing the temperature to a 3-D porous composite scaffold. This is illustrated in the middle steps of FIG. 1F.

As alternatives to nonsolvent-induced phase separation, additional methods can also be used to create porous scaffold structures via phase separation. These include, without limitation, thermally induced phase separation (TIPS) (see Nam et al., “Porous Biodegradable Polymeric Scaffolds Prepared by Thermally Induced Phase Separation,” J. Biomed. Mat. Res. 47(1):8-17 (1999); Lee et al., “Bicontinuous Phase Separation of Lithium-ion Battery Electrodes for Ultrahigh Areal Loading,” Proc. Natl. Acad. Sci. USA 117(35): 21155-21161 (2020), each of which is hereby incorporated by reference in its entirety); supercritical gel drying (see Cardea et al., “Supercritical Gel Drying: A Powerful Tool for Tailoring Symmetric Porous PVDF-HFP Membranes,” ACS Appl. Mater. Interfaces 1(1):171-180 (2009), which is hereby incorporated by reference in its entirety); and salt leaching (see Corriera et al., “Strategies for the Development of Three Dimensional Scaffolds from Piezoelectric Poly(vinylidene fluoride),” Materials & Design 92:674-681 (2016), which is hereby incorporated by reference in its entirety). Any other suitable phase separation techniques can also be utilized.

Subsequent to forming the scaffold, the mold is removed from the scaffold. Removal of the mold is preferably carried out by dissolving the mold from the exterior of the scaffold. Using PLA as the mold material, the PLA can be dissolved using chloroform to liberate the three-dimensional structure of the scaffold. This is illustrated in the final step of FIG. 1F.

Once the scaffold is liberated from the mold, the scaffold is then modified—as described above—to render the microchannels hydrophobic and/or the external surface of the scaffold hydrophilic. In addition, to facilitate hydrogel cross-linking to the hydrophilic external surface of the scaffold, the scaffold can optionally be treated with one or more agents to promote cross-linking of the scaffold surface to the hydrogel matrix. With the scaffold having these properties, the scaffold is ready to be integrated with the hydrogel matrix (containing one or more types of cells) to form the implantable cell containing devices. The scaffold can optionally be sterilized by any suitable means prior to use.

Having prepared the scaffold, the scaffold can be encapsulated by a hydrogel matrix (containing one or more cells) to form an implantable cell containing device of the invention. Briefly, a hydrogel precursor solution containing one or more cells suspended in the hydrogel precursor solution is formed, and then the hydrogel precursor solution is combined with the scaffold of the invention in a contained state, while allowing the hydrogel precursors to cross-link to form the hydrogel with the scaffold and the one or more cells embedded within the hydrogel.

The hydrogel layer is preferably formed using a cell growth matrix material. Suitable cell growth matrix materials include, without limitation, a natural polymeric material, a synthetic polymeric material, or a combination thereof.

Exemplary synthetic polymer materials include, without limitation, polyethylene glycol (PEG), poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly-2-methacryloyloxyethyl phosphorylcholine, N-hydroxyethyl acrylamide, copolymers thereof, derivatives thereof, and combinations thereof.

Exemplary natural polymeric materials include, without limitation, hyaluronate, collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, and derivatives or combinations thereof.

Preferred hydrogel materials include alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof. The alginate can be a pure alginate, a modified alginate, or a mixture of pure and modified alginate. Suitable modified alginates that can be used include zwitterionically modified alginate.

In any embodiment, the hydrogel layer has a thickness of about 100 μm to about 1000 μm. For example, and without limitation, the hydrogel layer of the implantable cell containing device may comprise a thickness of about 100 μm to about 200 μm, about 200 μm to about 300 μm, about 300 μm to about 400 μm, about 400 μm to about 500 μm, about 500 μm to about 750 μm, about 750 μm to about 1000 μm, about 100 μm to about 400 μm, about 100 μm to about 350 μm, about 100 μm to about 300 μm, about 150 μm to about 300 μm, or about 150 μm to about 300 μm.

The hydrogel layer comprises a preparation of cells. In any embodiment, the preparation of cells positioned or encapsulated in the hydrogel layer of the cell encapsulation device is a preparation of single cells such as a preparation of a single type of cells, and a preparation of multiple types of cells. In any embodiment, the preparation of a cells is a preparation of cell aggregates. In any embodiment, the preparation of cells is a preparation of single cells and cell aggregates.

The cells introduced into the hydrogel can be primary cells (autologous or allogenic) or immortalized cells.

In any embodiment, the preparation of cells can be mammalian cells, such as primate cells, rodent cells, canine cells, feline cells, equine cells, bovine cells, porcine cells, or human cells.

In certain embodiments, the preparation of cells positioned or encapsulated in the hydrogel layer includes stem cells or stem cell derived cells. The stem cells can be pluripotent, multipotent, oligopotent, or unipotent stem cells. For example, the preparation of stem cells may contain embryonic stem cells, epiblast cells, primitive ectoderm cells, primordial germ cells, and/or induced pluripotent stem cells.

In certain embodiments, the preparation of cells positioned or encapsulated in the hydrogel layer includes one or more of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme over-expressed feline kidney epithelial cells, myogenic cells, embryonic stem cell-derived neural progenitor cells, irradiated tumor cells, proximal tubule cells, neural precursor cells, astrocytes, genetically engineered cells.

In certain embodiments, the preparation of cells positioned or encapsulated in the hydrogel layer includes islet cells, such as a preparation of human islet cells, porcine islet cells, or rodent islet cells. The islet cells may produce insulin, glucagon, or both.

In certain embodiments, the preparation of cells positioned or encapsulated in the hydrogel layer includes cells that produce one or more of insulin, coagulation factors, albumin, urea, human cytochrome P450 enzymes, and combinations thereof.

In any embodiment, the cells may be present in the hydrogel layer at a cell density of between about 1×10³ to about 1×10¹⁰ cells/mL. For example, the cell density may range from about 1×10³ cells/mL up to about 1×10⁴ cells/mL, about 1×10⁴ cells/mL up to about 1×10⁵ cells/mL, about 1×10⁵ cells/mL up to about 1×10⁶ cells/mL, about 1×10⁶ cells/mL up to about 1×10⁷ cells/mL, about 1×10⁷ cells/mL up to about 1×10⁸ cells/mL, about 1×10⁸ cells/mL up to about 1×10⁹ cells/mL, or about 1×10⁹ cells/mL up to about 1×10¹⁰ cells/mL.

In any embodiment, the cells may be present in the hydrogel layer at a concentration of about 1%-40% v/v cells/hydrogel. For example, the cells are present in the hydrogel layer of the device at a concentration of about 5%-40% v/v, 10%-35% v/v, 15%-30% v/v, 20%-30% v/v, 10%-25% v/v, 15%-25% v/v, 5%-10% v/v, 5%-20% v/v, 5%-30% v/v, 35%-40% v/v, 30%-40% v/v, 25%-40% v/v, or 20%-40% v/v cells/hydrogel.

The hydrogel may optionally be supplemented with one or more cell factors or biologically active agents to enhance cell growth, differentiation, and/or survival of the cells positioned within the hydrogel material. Suitable biologically active agents include, without limitation, a protein, peptide, antibody or antibody fragment thereof, antibody mimetic, a nucleic acid, a small molecule, a hormone, a growth factor, an angiogenic factor, a cytokine, an anti-inflammatory agent, and any combination thereof.

Exemplary growth factors include, without limitation, an epidermal growth factor, fibroblast growth factor, transforming growth factor/bone morphogenetic protein, platelet derived growth factor, insulin growth factor, FGF, bFGF, acid FGF (aFGF), FGF-2, FGF-4, EGF, PDGF, TGF-beta, angiopoietin-1, angiopoietin-2, placental growth factor (PlGF), or VEGF.

Exemplary anti-inflammatory agents include, without limitation, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, tolmetin, and combinations thereof.

The hydrogel may optionally be supplemented with one or more contrast agents to facilitate in vivo monitoring of the implantable cell containing device when implanted to determine device placement, location of the implanted device at some time point after implantation, health of the implanted device, deleterious effects on non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and others known to those of skill in the art.

In certain embodiments, the preparation of cells positioned or encapsulated in the hydrogel layer includes islet cells that release insulin, glucagon, or both insulin and glucagon. In certain embodiments, the insulin producing cells may be a preparation of human SC-β cells. In certain embodiments, the preparation of islet cells and/or SC-β cells is a preparation of human islets and/or human SC-β cells, porcine islets and/or porcine SC-β cells, or rodent islets and/or rodent SC-β cells. In these embodiments, the preparation of cells comprises an islet density between about 1×10³ to about 6×10⁵ islet equivalents (IEQs)/mL. In these embodiments, the preparation of cells comprises an islet density between about 1×10³ to about 6×10⁴ islet equivalents (IEQs)/mL. For example, the islet equivalents may range from about 1×10³ up to about 2×10³, about 2×10³ up to about 3×10³, about 3×10³ up to about 4×10³, about 4×10³ up to about 5×10³, about 5×10³ up to about 6×10³, about 6×10³ up to about 7×10³, about 7×10³ up to about 8×10³, about 8×10³ up to about 9×10³, about 9×10³ up to about 1×10⁴, about 1×10⁴ up to about 2×10⁴, about 2×10⁴ up to about 3×10⁴, about 3×10⁴ up to about 4×10⁴, about 4×10⁴ up to about 5×10⁴, about 5×10⁴ up to about 6×10⁴, about 6×10⁴ up to about 7×10⁴, about 7×10⁴ up to about 8×10⁴, about 8×10⁴ up to about 9×10⁴, about 9×10⁴ up to about 1×10⁵, about 1×10⁵ up to about 2×10⁵, about 2×10⁵ up to about 3×10⁵, about 3×10⁵ up to about 4×10⁵, about 4×10⁵ up to about 5×10⁵, or about 5×10⁵ up to about 6×10⁵ islet equivalents (IEQs)/mL.

The implantable cell containing devices of the present invention are intended to be implanted in a subject to afford therapeutic benefit to that subject. In particular, the devices can be implanted using laproscopic surgical procedures or open surgical sites, and the devices can be placed subcutaneously, transcutaneously, preperitoneally, transperitoneally, or intraperitoneally. In some embodiments, implanting involves suturing the device or system to a body wall of the subject; anchoring the device to a body wall of the subject via a transabdominal portal; wrapping the delivery device or system in omentum of the subject; positioning the device in a cavity between the liver and the diaphragm; or anchoring the device to the diaphragm.

Both veterinary and medical uses are contemplated. Thus, exemplary subjects include, without limitation, a human, a mouse, a rat, a dog, a pig, a sheep, a cow, a horse, and a nonhuman primate.

By implanting the implantable cell containing devices, the devices can be used to deliver a therapeutic agent to a subject in need thereof. The treatment by implantation can be carried out for a limited duration over a period of days, weeks, or months. Thus, it is also contemplated that the method of treatment further involves retrieving the implantable cell containing device from the subject when no longer needed or when the device needs replacement, and optionally implanting a replacement implantable cell containing device after the initial device is retrieved.

As indicated above, the introduction of one or more contrast agents allows for monitoring of the device. Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implantable device. Monitoring of the device may be used to determine when to remove and replace a device, as necessary.

According to one embodiment, the subject has diabetes, is in need of diabetes treatment, and the method of delivering a therapeutic agent to the subject involves implanting an implantable cell containing device or system as described herein, which comprises a preparation of cells that release insulin, glucagon, or a combination thereof for the treatment of diabetes in the subject. Exemplary cells that release the therapeutic agent include one or more of islet cells, islets derived from a preparation of stem cells such as pluripotent, multipotent, oligopotent, or unipotent stem cells, including embryonic stem cells, epiblast cells, primitive ectoderm cells, primordial germ cells, and induced pluripotent stem cells.

According to one embodiment, the subject has a bleeding disorder, is in need of treatment for the bleeding disorder, and the method of delivering a therapeutic agent to the subject involves implanting an implantable cell containing device or system as described herein, which comprises a preparation of cells that release a therapeutic agent that treats the bleeding disorder. In accordance with this embodiment, the bleeding disorder can be any bleeding disorder, such as hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency, and the therapeutic agent is a blood clotting factor selected from the group of Factor I, Factor II, Factor V, Factor VII, Factor VIII, Factor IX, Factor X, Factor XI, Factor XII, Factor XIII, and combinations thereof. Exemplary cells that release the therapeutic agent include one or more of recombinant myoblasts, mesenchymal stromal cells, endothelial cells, induced pluripotent stem cell derived endothelial cells, induced pluripotent stem cell derived mesenchymal stromal cells, and a combination thereof.

In another embodiment, the subject has a lysosomal storage disorder, is in need of treatment for the lysosomal storage disorder, and the method of delivering a therapeutic agent to the subject involves implanting an implantable cell containing device or system as described herein into the subject having the lysosomal storage disorder. In accordance with this embodiment, the therapeutic agent is an enzyme selected from the group of α-L-iduronidase, Iduronate-2-sulfatase, α-glucuronidase, Arylsulfatase A, alpha-Galactosidase A, and combinations thereof. Exemplary cells that release the therapeutic agent include one or more of hematopoietic stem cells, fibroblasts, myoblasts, Baby Hamster Kidney (BHK) cells, Chinese Hamster Ovary cells, Human Amniotic Epithelial (HAE) cells, mesenchymal stromal cells, induced pluripotent stem cell derived mesenchymal stromal cells, and combinations thereof.

According to one embodiment, the subject has a neurological disorder, is in need of treatment for the neurological disorder, and the method of delivering a therapeutic agent to the subject involves implanting an implantable cell containing device or system as described herein, which comprises a preparation of cells that release a therapeutic agent that treats the neurological disorder. In accordance with this embodiment, the neurological disorder is Parkinson's disease, Alzheimer's disease, epilepsy, Huntington's disease, Amyotrophic lateral sclerosis, chronic pain, a sensory disorder such as visual loss, hearing loss, peripheral nerve injury, and spinal cord injury, and the therapeutic agent is selected from the group of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof. Exemplary cells that release the therapeutic agent include one or more of choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, myogenic cells, embryonic stem cell-derived neural progenitor cells, and combinations thereof.

In another embodiment, the subject has a cancerous condition, is in need of treatment for the cancerous condition, and the method of delivering a therapeutic agent to the subject involves implanting an implantable cell containing device or system as described herein into the subject having the cancerous condition. In accordance with this embodiment, the therapeutic agent is one or more of IL-2, endostatin, cytochrome P450 enzyme, a tumor antigen, a cytokine, and combinations thereof. Exemplary cells that release the therapeutic agent include one or more of IL-2-secreting myoblasts, endostatin-secreting cells, Chinese Hamster Ovary cells, cytochrome P450 enzyme overexpressed feline kidney epithelial cells, irradiated tumor cells, and combinations thereof.

In another embodiment, the subject has a chronic eye disease, is in need of treatment for the chronic eye disease, and the method of delivering a therapeutic agent to the subject involves implanting an implantable cell containing device or system as described herein into the subject having the chronic eye disease. The chronic eye disease may be any one of age-related macular degeneration, diabetic retinopathy, retinitis pigmentosa, glaucoma, macular telangiectasia, and combinations thereof. In accordance with this embodiment, the therapeutic agent is one or more trophic factors that protect compromised retinal neurons and restore neural circuits, such as any one or more of ciliary neurotrophic factor, antagonists against vascular endothelial growth factor and platelet-derived growth factor, and combinations thereof. Exemplary cells that release the therapeutic agent include one or more of human retinal pigment epithelium cells, recombinant human retinal pigment epithelium cells, and combinations thereof.

In one embodiment, the subject has kidney disease (kidney failure), is in need of treatment for the kidney disease (kidney failure), and the method of delivering a therapeutic agent to the subject involves implanting an implantable cell containing device or system as described herein into the subject having the kidney disease (kidney failure). In accordance with this embodiment, the therapeutic agent is dopamine, atrial natriuretic peptide, and combinations thereof. Exemplary cells that release the therapeutic agent include one or more of renal proximal tubule cells, mesenchymal stem cells, and combinations thereof.

In one embodiment, the subject has chronic pain, is in need of treatment for the chronic pain, and the method of delivering a therapeutic agent to the subject involves implanting an implantable cell containing device or system as described herein into the subject having the chronic pain. The chronic pain can be any chronic pain condition including, without limitation, those caused by degenerative back and knee, neuropathic back and knee, or cancer. In accordance with this embodiment, the therapeutic agent is catecholamine, opioid peptides, enkephalins, and combinations thereof. Exemplary cells that release the therapeutic agent include one or more of chromaffin cells, neural precursor cells, mesenchymal stem cells, astrocytes, and genetically engineered cells, and combinations thereof.

Wherever the word “about” is employed herein in the context of dimensions (e.g. distances, sizes), time, amounts (relative amounts, concentration, etc.), cell densities, etc., it will be appreciated that such variables are approximate and as such may vary by ±10%, for example ±5% and preferably ±2% (e.g. ±1%) from the numbers specified herein.

### EXAMPLES

The examples below are intended to exemplify the practice of embodiments of the disclosure but are by no means intended to limit the scope thereof.

### Materials & Methods for Examples 1-5

Materials: Poly(vinylidene fluoride-co-hexafluoropropylene) (PVDF-HFP, Mw=455 kDa), Tris hydrochloride (Tris-HCl), sodium hydroxide, dopamine hydrochloride, sodium chloride (NaCl), calcium chloride dihydrate (CaCl_{2·}2H₂O), barium chloride dihydrate (BaCl_{2·}2H₂O), calcium sulfate dihydrate (CaSO₄·2H₂O), Nile Red, gelatin, and D-glucose were purchased from Sigma-Aldrich. Poly(lactic acid) (PLA) filament was purchased from PRUSA. Ultrapure sodium alginate (Pronova SLG100) was purchased from NovaMatrix. Water was deionized to 18.2 MΩ·cm with a Synergy UV purification system (Millipore Sigma).

Animals: Male C57BL/6J mice (2 months old) were purchased from The Jackson Laboratory. The mice were maintained at a temperature of 70-72° F. with 30-70% humidity under a 14-hour light/10-hour dark cycle. Male Sprague-Dawley rats (weight of ˜300 g) were purchased from Charles River Laboratories.

Characterizations: High-resolution X-ray computer tomography (Nano-CT) scanning was conducted on a 3D X-ray microscope (ZEISS Xradia 520 Versa). Scanning electron microscopy (SEM) and energy dispersive X-ray spectroscopy (EDS) element mapping were performed using a field emission scanning electron micro-analyzer (LEO 1550). Contact angle images were taken using a contact angle goniometer (Rame-Hart 500). Optical and fluorescent microscope images were taken using a digital microscope (EVOS FL). H&E staining images were taken using an Aperio Scanscope (CS2). Stereo microscope images were taken by a stereomicroscope (Olympus SZ61). Immunofluorescence images were taken using a confocal microscope (ZEISS LSM 710). OriginPro 8.5.1 software and GraphPad Prism 8 software were used for data plotting.

Fabrication of the SONIC Scaffold: PVDF-HFP was dissolved in acetone at concentration of 15 wt % under heat in a sealed glass vial. After cooling to room temperature, the PVDF-HFP solution was filled into a 3D printed PLA mold (Original Prusa i3 MK2S) and, after ˜1 minute, the polymer solution and mold were immersed in a water/ethanol (V/V=1/1) bath for ˜10 minutes to facilitate the phase separation process, and then transferred to a water bath for ˜30 minutes for a solidification process. Next, the solidified PVDF-HFP was immersed in ethanol and hexane for two dehydration steps of about ˜10 minutes each, followed by air drying at ambient temperature. Finally, the SONIC scaffold was obtained after the selective extraction of the PLA mold with chloroform. The SONIC scaffolds used for in vivo studies were sterilized by autoclave prior to use.

Nano-CT Imaging of the Mealworm and SONIC Scaffold: To prepare a mealworm specimen for Nano-CT scanning, a 2 cm-long mealworm was loaded in a 1 mL pipet tip and sacrificed by freezing at −20° C. 6 individual scans were performed on different sections of the mealworm using an “oversize scan” option to get a full image of the specimen. During the scans, the X-ray source was set to a voltage of 100 kV, and the scanning resolution was set as 5.19 μm per pixel under a binning mode of 2×2. Subsequently, 3D reconstruction of the obtained images was performed using Avizo software (version 8.1.1). A segmentation process was conducted to visualize the tracheal system of the mealworm based on the different absorption contrasts between the respiratory gases and mealworm tissues.

To prepare a SONIC scaffold specimen for the Nano-CT scanning, a small piece of scaffold (˜1 mm³) was cut and attached on a tip. During the scan, the X-ray source was set to a voltage of 100 kV, and the scanning resolution was set as 0.268 μm per pixel under a binning mode of 2×2. Subsequently, 3D reconstruction of the obtained images was performed using Avizo software. Network connectivity on the polymeric and the porous regions of the scaffold was performed using ImageJ.

Electron Paramagnetic Resonance (EPR) for O₂ Mapping: O₂ mapping was performed on a 25 mT EPR imager (JIVA-25, O2M Technologies, LLC). The JIVA-25 operates at 720 MHz using electron paramagnetic resonance oxygen imaging (EPROI) principles and utilizes oxygen sensitive electron spin-lattice relaxation rates (T₁) of trityl radical probe OX063-D24 (methyl-tris[8-carboxy-2,2,6,6-tetrakis[(2-hydroxyethyl]benzo[1,2-d:4,5-d′]bis[1,3]dithiol-4-yl]-trisodium salt) for reporting pO₂.

A SONIC scaffold or control scaffold was fixed at the bottom of a glass tube (VWR, 10×75 mm) using a dental vinyl polysiloxane impression material. Gelatin solution (1 mL) (1 wt %) containing spin probe (1 mM OX063-d24) was filled into the container with the top end of scaffold exposed above the gelatin. First, the system was deoxygenated using N₂ to reduce pO₂ close to 0 mmHg. After deoxygenation, the system was exposed to a gas mixture containing 5% O₂ and 95% N₂, and the pO₂ change in gelatin was continuously monitored until a steady state (40 mmHg) was approached.

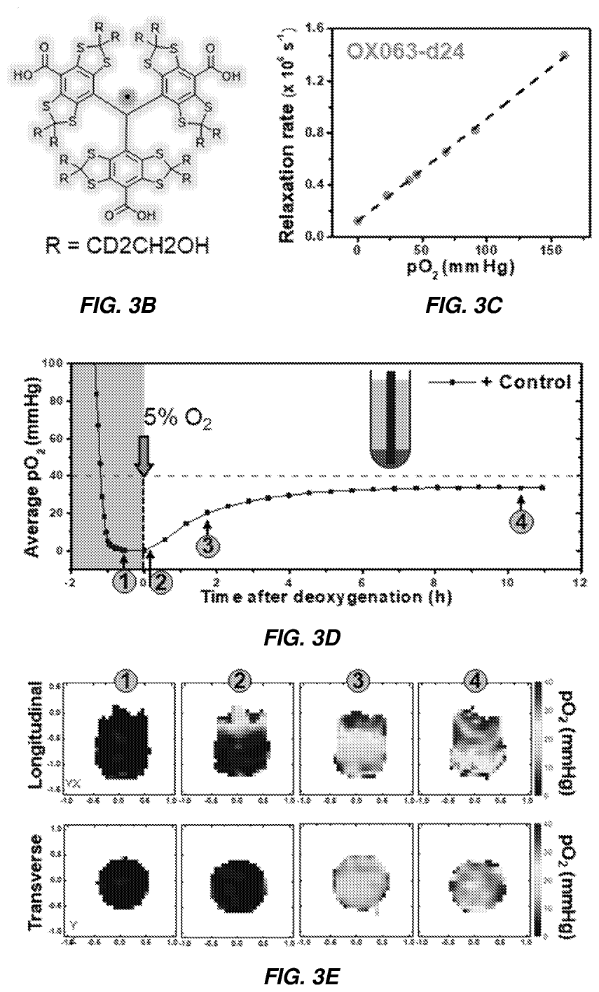

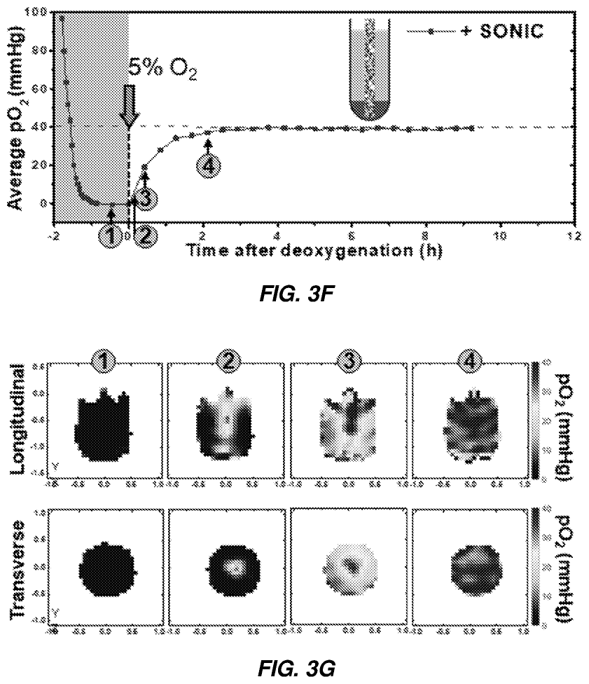

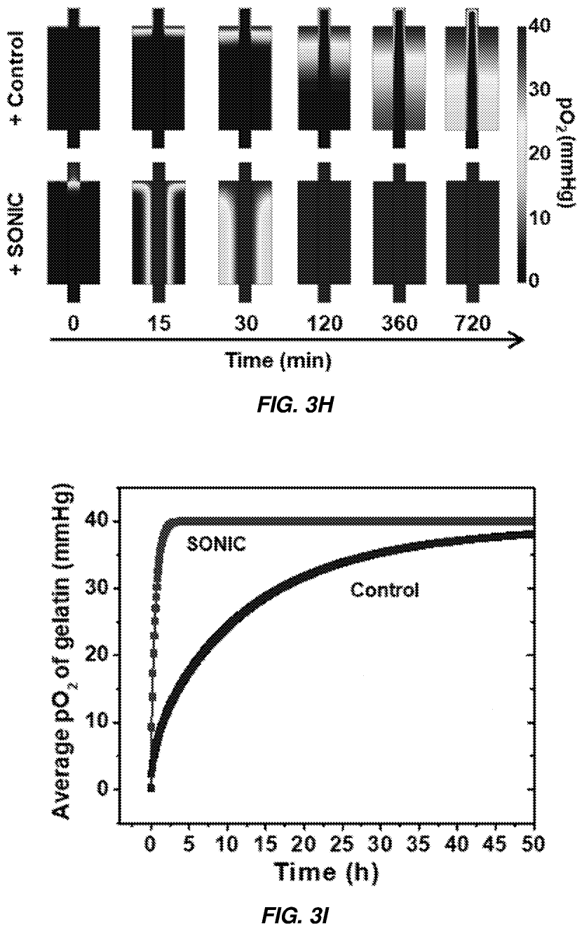

Average pO₂ measurements were performed using inversion recovery electron spin echo (IRESE) sequence (Epel et al., “Absolute Oxygen R1e Imaging In Vivo with Pulse Electron Paramagnetic Resonance,” Magnetic Resonance in Medicine 72:362-368 (2014), which is hereby incorporated by reference in its entirety) with the following parameters: pulse lengths 60 ns, 16 phase cycles scheme with FID suppression, spin echo delay 500 ns, 80 logarithmically spaced delays from 400 ns-65 μs, 100 us repetition time. The curves were fitted using single exponential recovery to extract R₁ (1/T₁) values that were converted to pO₂ (FIG. 3C). pO₂ imaging was performed using IRESE sequence with the following parameters: pulse lengths 60 ns, 16 phase cycles scheme with FID suppression, spin echo delay 400 ns, equal solid angle spaced 654 projections, 67 baselines, 1.5 G/cm gradient, 10 time delays from 410 ns-40 μs, 35 μs-59 μs repetition time, overall 10 min image duration. Images were reconstructed using filtered back-projection in isotropic 64×64×64 cube with 0.66 mm voxel linear size.

Fabrication of the SONIC Device: The SONIC scaffold was immersed into a dopamine solution (2 mg/mL in 10 mM tris buffer, pH 8.5) overnight to create a hydrophilic polydopamine coating on the scaffold surface. Subsequently, CaSO₄ was deposited onto the scaffold surface by dipping it into a CaSO₄ saturated solution (0.24 wt % in water) and then drying it at 60° C., leaving CaSO₄ crystals on the scaffold surface. Next, the scaffold was inserted into a glass tubing mold with sodium alginate (2%) solution. Alginate crosslinking then occurred by the Ca²⁺ ions diffused from the CaSO₄. Finally, the SONIC device was pushed out from the tubing mold into a cross-linking buffer (95 mM CaCl₂+5 mM BaCl₂), leaving the device in buffer around 4 min for a further cross-linking. Constructs which contained INS-1 cells or islets were fabricated by premixing the alginate solution with the cells before application onto the scaffolds.

To fabricate control devices without the SONIC scaffold, a tubing mold was prepared by rolling a dialysis membrane (Spectra/Por®, MWCO 3500) into a tube with an inner diameter of ˜4 mm and sealing one end with a PDMS cap. Then, INS-1 cells or islets alginate solution were loaded into the mold and immersed in the buffer (95 mM CaCl₂+5 mM BaCl₂) for cross-linking by the Ca²⁺ and Ba²⁺ ions which diffused through the dialysis membrane. Next, the tubing mold was unrolled to leave the alginate in the buffer around 4 min for further cross-linking.

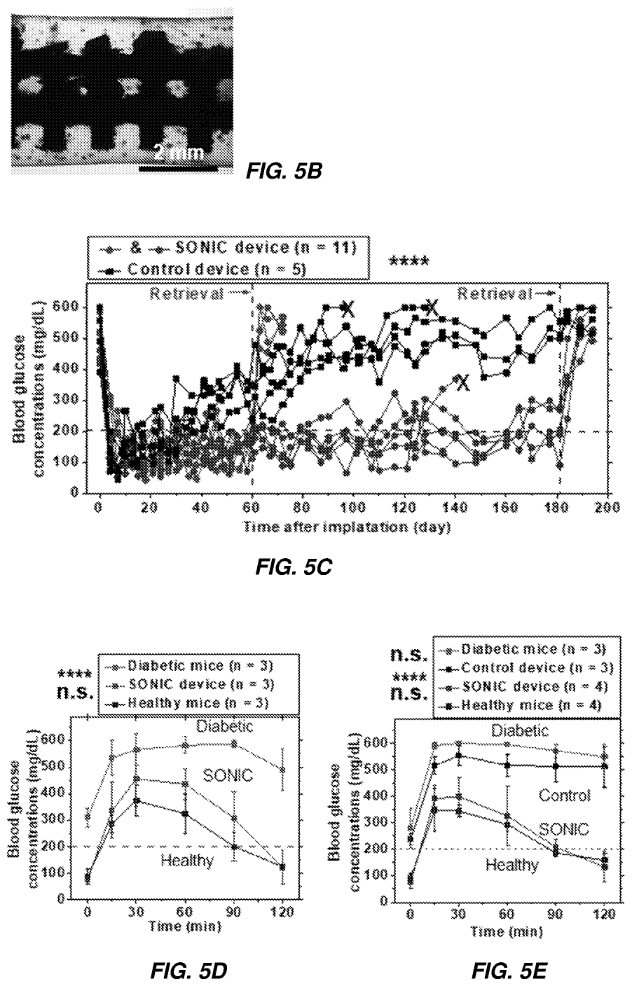

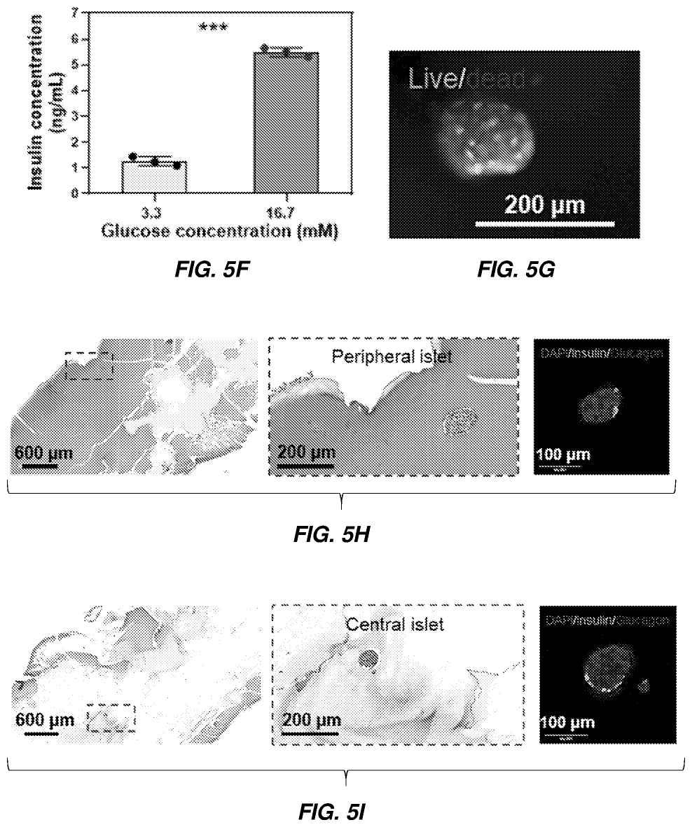

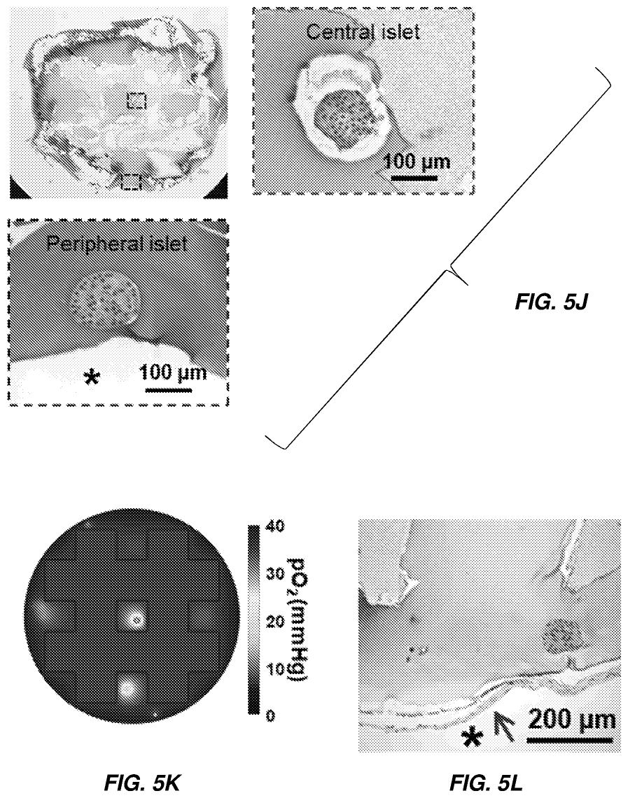

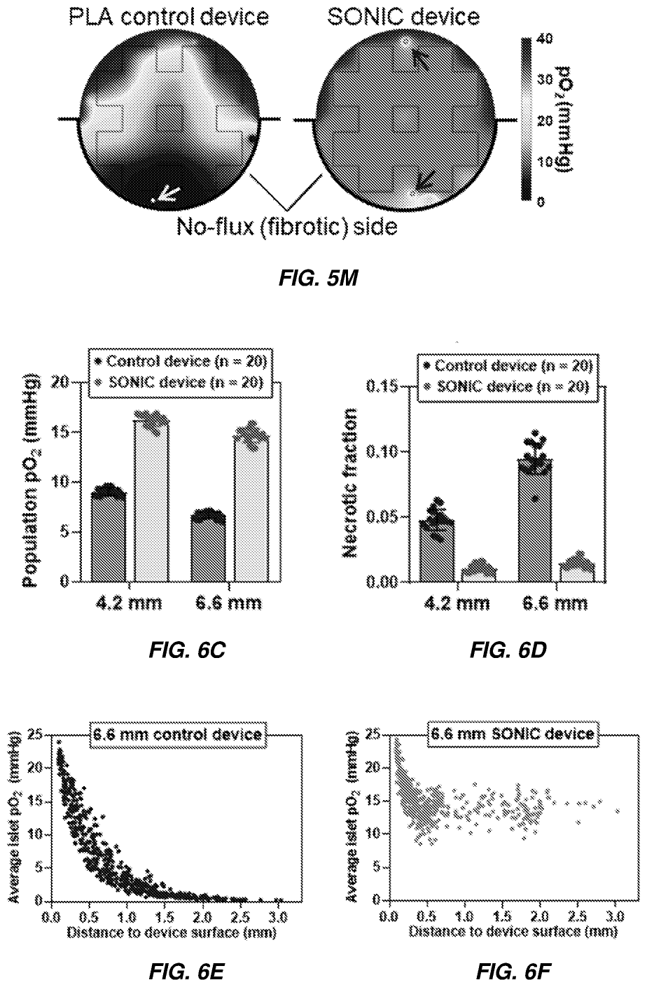

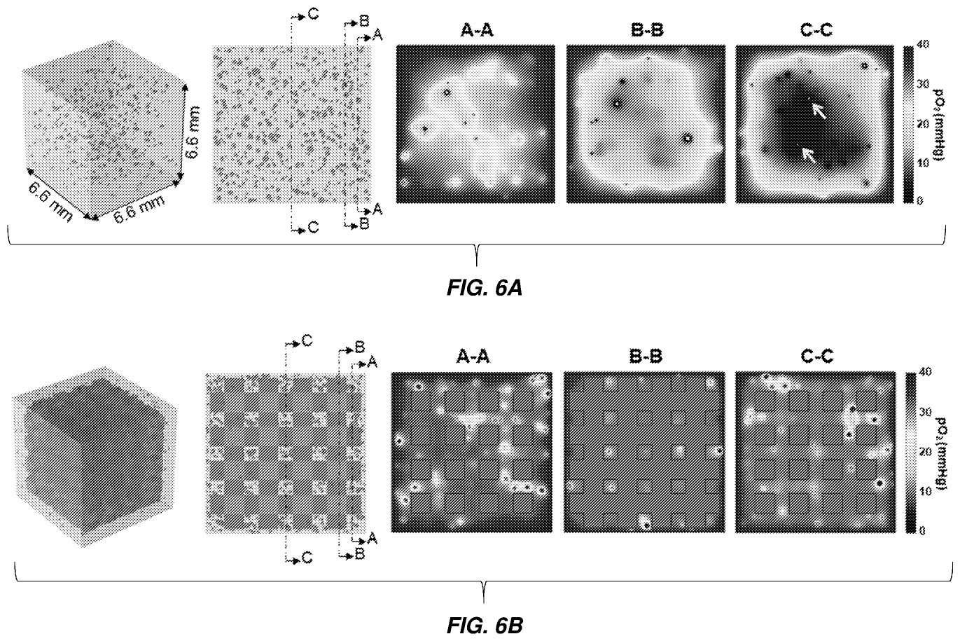

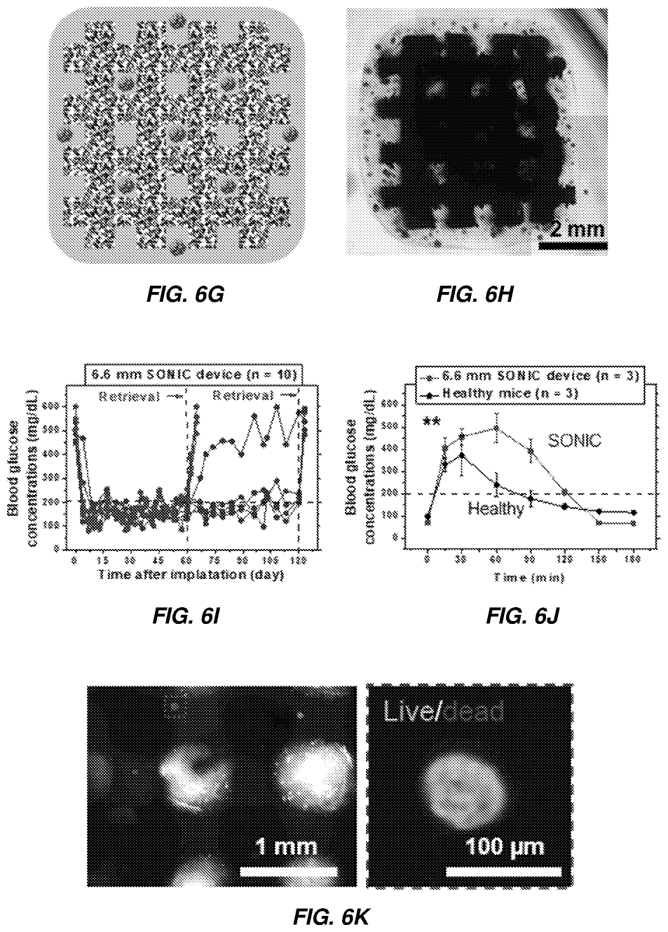

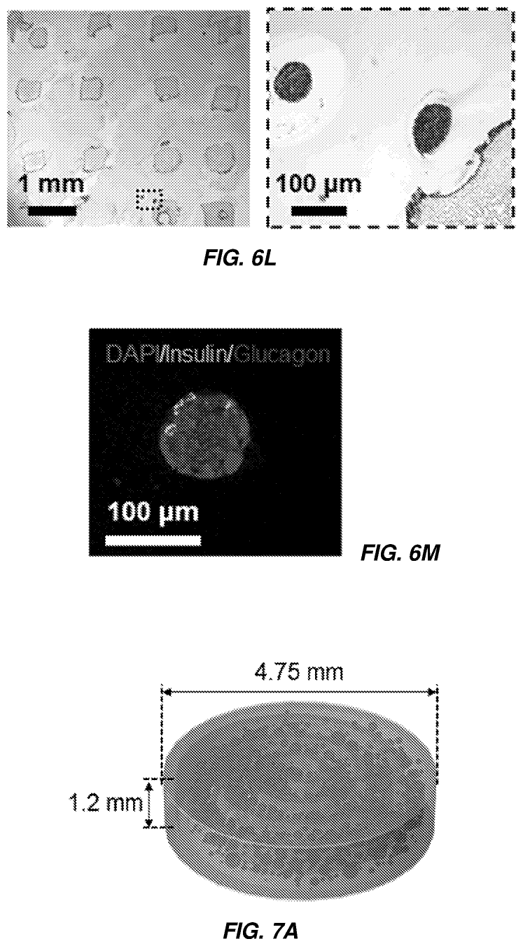

For the devices in mice studies, 500 IEQ of rat islets distributed in approximately 170 μL alginate were incorporated in each cylindrical (4.2 mm in diameter, 20.4 mm in length) SONIC device (FIG. 5B) and 500 IEQ of rat islets distributed in approximately 280 μL alginate were incorporated in each corresponding cylindrical scaffold-free control device; 500 IEQ of rat islets distributed in approximately 160 μL alginate were incorporated in each cubic (6.6×6.6×6.6 mm) SONIC device (FIG. 6H).

In Vitro Cell Viability Study: INS-1 cells were purchased from Sigma-Aldrich and cultured with RPMI 1640 medium (Gibco) supplemented with 10% FBS (Gibco), 10 mM HEPES (Gibco), 2 mM glutamine (Gibco), 1 mM sodium pyruvate (Gibco), 50 μM β-mercaptoethanol (Gibco), and 1% penicillin/streptomycin (Gibco). Trypsin-dissociated INS-1 cells were suspended in alginate solution at a density of 2.5 million cells/mL and incorporated into SONIC devices or control devices, and then were incubated in the above-mentioned medium in a hypoxic incubator with 5% O₂, 5% CO₂ at 37° C. After 48 hours, the cells in devices were stained with a LIVE/DEAD™ viability/cytotoxicity kit (Invitrogen).

Rat Islet Isolation and Purification: Sprague-Dawley rats were used for harvesting islets. The rats were anesthetized using 3% isoflurane in O₂ throughout the whole surgery. Briefly, the pancreas was distended with 10 mL 0.15% Liberase (Roche) in M199 media (Gibco) through the bile duct. The pancreas was digested at 37° C. circulating water bath for ˜28 mins (digestion time varied slightly for different batches of Liberase). The digestion was stopped by adding cold M199 media with 10% FBS (Gibco). After vigorously shaking, the digested pancreases were washed twice with media (M199+10% FBS), filtered through a 450 μm sieve, and then suspended in a Histopaque 1077 (Sigma)/M199 media gradient and centrifuged at 1700 RCF with 0 break and 0 acceleration for 17 min at 4° C. This gradient centrifugation step was repeated for higher purity. Finally, the islets were collected from the gradient and further isolated by a series of gravity sedimentations, in which each top supernatant was discarded after 4 min of settling. Islet equivalent (IEQ) number of purified islets was counted by reported IEQ conversion factors (Buchwald et al., “Quantitative Assessment of Islet Cell Products: Estimating the Accuracy of the Existing Protocol and Accounting for Islet Size Distribution,” Cell Transplant 18:1223-1235 (2009), which is hereby incorporated by reference in its entirety). Islets were then washed once with islet culture media (RPMI 1640 supplemented with 10% FBS, 10 mM HEPES, and 1% penicillin/streptomycin) and cultured in this medium overnight before further use.

Implantation and Retrieval in Mice: C57BL/6J mice were administered an intraperitoneal injection of freshly prepared STZ solution (22.5 mg/mL in 100 mM sodium citrate buffer, pH 4.5) at a dosage of 150 mg STZ/kg mouse to induce diabetes one week before device implantation. Only mice with non-fasted blood glucose levels above 350 mg/dL were considered as diabetic. The diabetic mice were anesthetized with 3% isoflurane in O₂ and the abdomen area was shaved and sterilized using betadine and 70% ethanol. A small skin incision (˜8 mm) was made along the midline of the abdomen, and then a following incision was made along the linea alba. The device was introduced into the peritoneal cavity through the incision. The peritoneal wall was closed using 5-0 absorbable polydioxanone (PDS II) sutures and the skin incision was closed using 5-0 nylon sutures.

For retrieval, the mice were treated with the same procedures as above. Then, the device was located and pulled out from the peritoneal cavity using a tweezer. The incisions were sutured and keep the mice alive for following BG monitoring after device retrieval.

Morphology and Immunohistochemistry of Islets in Retrieved Devices: The retrieved devices were fixed with 10% formalin, embedded in paraffin, and sectioned into 5 μm sections. Hematoxylin and eosin (H&E) staining was performed. For immunofluorescent insulin and glucagon staining, paraffin-embedded sections were deparaffinized in xylene and sequentially rehydrated in 100% ethanol, 95% ethanol, 75% ethanol, and PBS. Slides were then boiled in citric acid buffer (10 mM citric acid, 0.05% Tween 20, pH 6.0) for 30 min for antigen retrieval. After blocking with 5% donkey serum, primary rabbit anti-rat insulin (Abcam, ab63820, 1:200), and mouse anti-rat glucagon (Abcam, ab10988, 1:200) antibodies were applied and incubated overnight at 4° C. After washing with PBS, Alexa Fluor 594-conjugated goat anti-rabbit IgG (Thermofisher, A11037, 1:400) and Alexa Fluor 488-conjugated donkey anti-mouse IgG (Thermofisher, A21202, 1:400) were applied and incubated for 60 min. Finally, slides were washed with PBS, applied with antifade/DAPI, and covered with glass coverslips.

BG Monitoring and Intraperitoneal Glucose Tolerance Test (IPGTT): Mouse BG levels were measured by a commercial glucometer (Contour Next EZ, Bayer) with a drop of blood collected from the tail vein. For the IPGTT, mice were fasted for 16 hours and an intraperitoneal injection of 20% glucose solution was administered at a dosage of 2 g glucose/kg mouse. BG levels were measured at 0, 15, 30, 60, 90, and 120 min following the injection.

Ex Vivo Static Glucose-Stimulated Insulin Secretion (GSIS) Assay: Krebs Ringer Bicarbonate (KRB) buffer was prepared as follows: 98.5 mM NaCl, 4.9 mM KCl, 2.6 mM CaCl₂·2H₂0, 1.2 mM MgSO₄·7H₂O, 1.2 mM KH₂PO₄, 25.9 mM NaHCO₃, 0.1% BSA (all from Sigma-Aldrich), and 20 mM HEPES (Gibco). The retrieved devices were incubated in the KRB buffer for 2 hours at 37° C., 5% CO₂. Devices were transferred and incubated in KRB buffer supplemented with 3.3 mM glucose, then 16.7 mM glucose for 75 min each. The buffer was collected after each incubation step, and insulin concentration was measured using an ultrasensitive rat insulin ELISA kit (ALPCO).

Computational Modeling: Five general finite element models were created to calculate theoretical O₂ profiles in SONIC-enabled constructs and corresponding controls. In all models, O₂ tension (pO₂) was related to the concentration of O₂ (cₒ_{₂}) by the Bunsen solubility, or equilibrium concentration of O₂ in a material i at 37° C., αₒ_{₂}_{,i}:

$${c}_{{O}_{2}} = {α}_{{O}_{2} , i} \cdot p {O}_{2} \qquad ( 1 )$$

In other words, O₂ partitioning was governed by Henry's law where, in Equation 1, αₒ_{₂}_{,i} represents the inverse of Henry's constant. Each model is described below.

Model 1 (FIGS. 3H-I) simulated time-dependent oxygenation of the in vitro acellular test. Two domains were considered: a rectangular prism representing the SONIC scaffold or PLA control (2×2×23 mm), and a surrounding cylinder (8.6 mm diameter, 17 mm length), representing gelatin, with the scaffold located in the center of the gelatin. To emulate the experimental set up, boundary conditions at the top face of the gelatin and exposed faces of the scaffold were implemented at a constant pO₂ of 40 mmHg, while no flux conditions were implemented on all other faces due to the O₂ impermeability of the glass test tube and fixing resin containing the system. Transient pO₂ transport in each domain was governed by Fick's second law:

$${α}_{{O}_{2} , i} \frac{\partial ( p {O}_{2} )}{\partial t} = - {α}_{{O}_{2} , i} {D}_{{O}_{2} , i} ( \frac{{\partial }^{2} ( p {O}_{2} )}{\partial {x}^{2}} + \frac{{\partial }^{2} ( p {O}_{2} )}{\partial {y}^{2}} + \frac{{\partial }^{2} ( p {O}_{2} )}{\partial {z}^{2}} ) \qquad ( 2 )$$

Here, Dₒ_{₂}_{,i} represents diffusivity of O₂ in domain i (i.e., SONIC scaffold, PLA, or gelatin) at 37° C. As it was confirmed that a bicontinuous porous structure was maintained throughout the SONIC scaffold (FIGS. 1J-K), this domain was modeled as a gaseous air phase. Therefore, αₒ_{₂}_{,SONIC}=3.9×10⁻⁴ mol/(m³ Pa) rather represents the equilibrium O₂ concentration in air and was calculated by the ideal gas law. Solubility and diffusivity values for gelatin varied widely in the literature, thus values for alginate hydrogel were used, given their physical and chemical similarity. The remaining solubilities were thus implemented as αₒ_{₂}_{,gelatin}=αₒ_{₂}_{,alginate}=9.3×10⁻⁶ mol/(m³ Pa) (Lewis, “Eliminating Oxygen Supply Limitations for Transplanted Microencapsulated Islets in the Treatment of Type 1 Diabetes,” Thesis (2008), which is hereby incorporated by reference in its entirety), and αₒ_{₂}_{,PLA}=4.5×10⁻⁵ mol/(m³ Pa) (Courgneau et al., “Analysis of the Structure-Properties Relationships of Different Multiphase Systems Based on Plasticized Poly(Lactic Acid),” Journal of Polymers and the Environment 19:362-371 (2011), which is hereby incorporated by reference in its entirety), each obtained from the literature. Likewise, Dₒ_{₂}_{,SONIC}=1.8×10⁻⁵ m²/s (Chapman et al., “The Mathematical Theory of Non-Uniform Gases: An Account of the Kinetic Theory of Viscosity, Thermal Conduction and Diffusion in Gases,” Cambridge University Press (1990), which is hereby incorporated by reference in its entirety), Dₒ_{₂}_{,gelatin}=Dₒ_{₂}_{,alginate}=2.7×10⁻⁹ m²/s (Lewis A S., “Eliminating Oxygen Supply Limitations for Transplanted Microencapsulated Islets in the Treatment of Type 1 Diabetes,” Thesis (2008); Mehmetoglu et al., “Oxygen Diffusivity in Calcium Alginate Gel Beads Containing Gluconobacter Suboxydans,” Artificial Cells, Blood Substitutes, and Biotechnology 24:91-106 (1996), which are hereby incorporated by reference in their entirety)^{59,65}, Dₒ_{₂}_{,PLA}=1.6×10⁻¹² m²/s (Courgneau et al., “Analysis of the Structure-Properties Relationships of Different Multiphase Systems Based on Plasticized Poly(Lactic Acid),” Journal of Polymers and the Environment 19:362-371 (2011), which is hereby incorporated by reference in its entirety), also obtained from the literature, were implemented for O₂ diffusivities in the respective materials. A time dependent study simulated over 80 hours was performed with a time step of 5 mins.

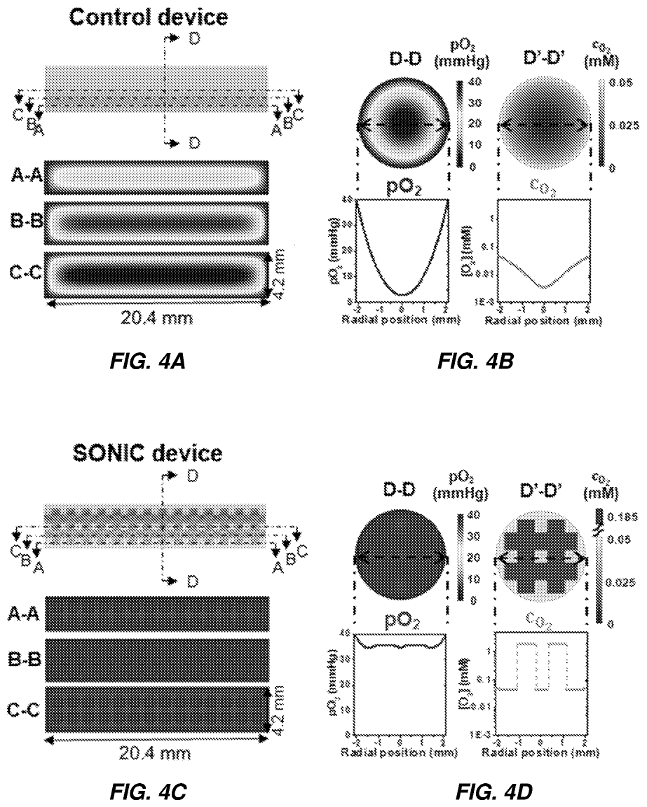

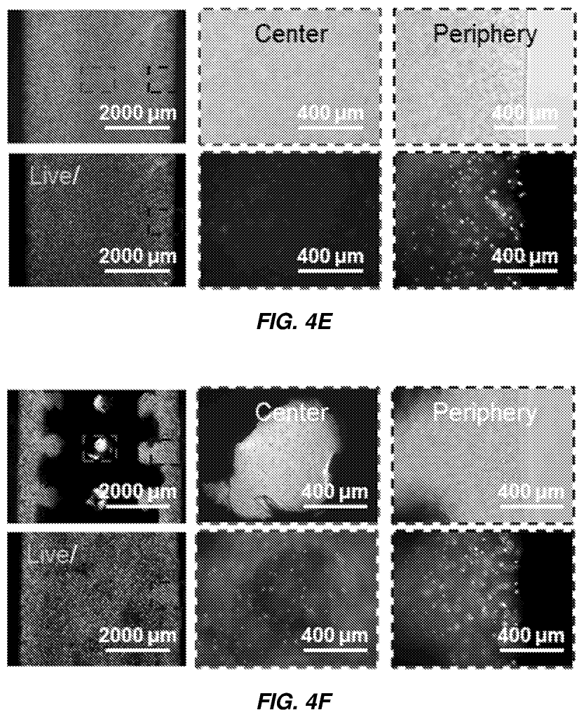

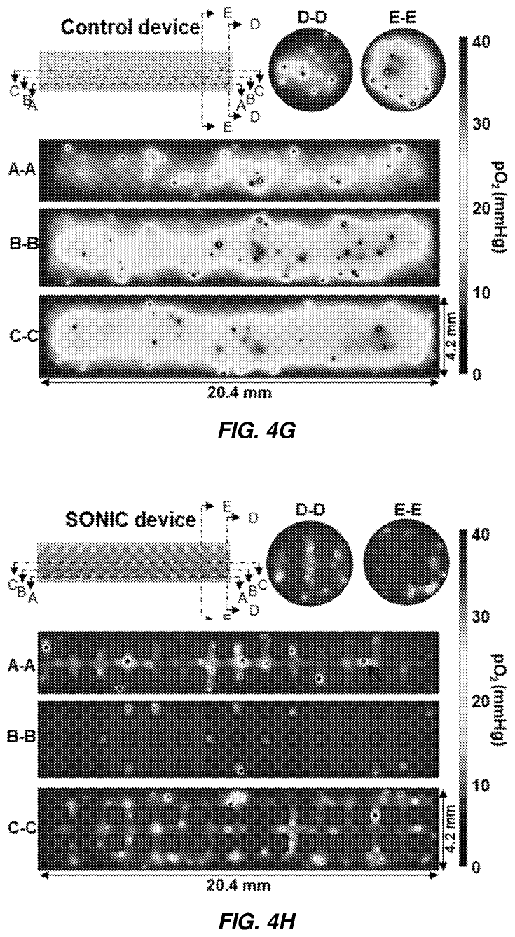

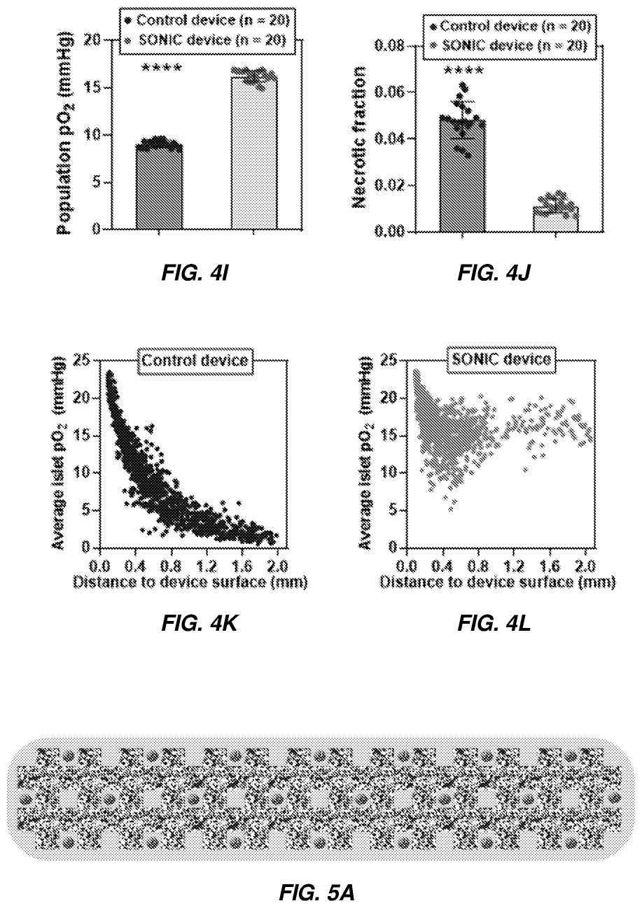

Model 2 simulated steady state O₂ transport in cylindrical constructs (4.2 mm diameter, 20.4 mm length) containing alginate-encapsulated INS-1 cells with cell densities from 1.0 to 8.0 million cells per mL alginate (FIGS. 4A-D). O₂ profiles in a construct featuring the ladder-like SONIC scaffold were compared to those in a scaffold-free control. The alginate and encapsulated cells were modeled as one composite domain, with diffusivity and solubility coefficients of that of alginate. A constant pO₂ of 40 mmHg was implemented at all external boundaries. Steady state pO₂ profiles were obtained by solving the diffusion-reaction mass balance equation:

$${α}_{{O}_{2} , i} {D}_{{O}_{2} , i} ( \frac{{\partial }^{2} ( p {O}_{2} )}{\partial {x}^{2}} + \frac{{\partial }^{2} ( p {O}_{2} )}{\partial {y}^{2}} + \frac{{\partial }^{2} ( p {O}_{2} )}{\partial {z}^{2}} ) = - {R}_{{O}_{2}} \qquad ( 3 )$$

Above, R₀₂ represents O₂ consumption by the encapsulated INS-1 cells, modeled using Michaelis-Menten kinetics and a step-down function (An et al., “An Atmosphere-Breathing Refillable Biphasic Device for Cell Replacement Therapy,” Advanced Materials 31:1905135 (2019), which is hereby incorporated by reference in its entirety):

$${R}_{{O}_{2}} ( p {O}_{2} ) = \{ \begin{matrix}0 & , p {O}_{2} < 0.08 mmHg \\ \frac{{V}_{INS - 1} \cdot ρ}{{α}_{{O}_{2} , alginate}} \cdot ( \frac{p {O}_{2}}{{p O}_{2} + {K}_{m}} ) & , p {O}_{2} \geq 0.08 mmHg\end{matrix} \qquad ( 4 )$$

In Equation 4, V_{INS-1}=5.0×10⁻¹⁷ mol/(m³ s cell) represents the literature-retrieved INS-1 cellular O₂ consumption rate (Cline et al., “Rates of Insulin Secretion in INS-1 Cells are Enhanced by Coupling to Anaplerosis and Kreb's Cycle Flux Independent of ATP Synthesis,” Biochemical and Biophysical Research Communications 415:30-35 (2011), which is hereby incorporated by reference in its entirety), Kₘ=0.81 mmHg represents the half-maximum constant derived from studies on mitochondrial respiration (Wilson et al., “The Oxygen Dependence of Mitochondrial Oxidative Phosphorylation Measured by a New Optical Method for Measuring Oxygen Concentration,” J Biol. Chem. 263:2712-2718 (1988), which is hereby incorporated by reference in its entirety), and ρ represents the cell density which was implemented at 2.5 million cells/mL to match experimental conditions (FIGS. 4A-F) but also varied between 1-8 million cells/mL to explore the effect of varying cell density. Below the threshold of pO₂=0.08 mmHg, O₂ consumption is set to zero (Buchwald P. “FEM-Based Oxygen Consumption and Cell Viability Models for Avascular Pancreatic Islets,” Theoretical Biology and Medical Modelling 6:5 (2009); Buchwald et al., “Glucose-Stimulated Insulin Release: Parallel Perifusion Studies of Free and Hydrogel Encapsulated Human Pancreatic Islets,” Biotechnology and Bioengineering 115:232-245 (2018), which are hereby incorporated by reference in their entirety), representing the lack of respiration in necrotic cells as in models described elsewhere.

Model 3 simulated steady state O₂ transport in all cylindrical constructs (4.2 mm in diameter, 20.4 mm or 6.4 mm in length) containing alginate-encapsulated rat islets (FIGS. 4G-L, 5K, and 5M)(500 IEQ rat islets per device) or human islets with cell densities from 2.74% to 8.04% v/v in devices (4.2 mm in diameter, 2.2 mm in length). In all cases, islets were implemented as perfect spheres and seeded randomly in the alginate domain. As a default, islet diameters, d, were randomly selected from a size distribution. Rat islet diameters were selected from a lognormal distribution, with the probability density function given by:

$$f ( d ) = \frac{1}{d α \sqrt{2 π}} exp ( - \frac{{ln ( d / β )}^{2}}{2 {α}^{2}} ) \qquad ( 5 )$$

where α=0.40 and β=112.6. These values were obtained empirically and were found to be similar to distributions observed in other animal islet sources (Jo et al., “Size Distribution of Mouse Langerhans Islets,” Biophysical Journal 93:2655-2666 (2007), which is hereby incorporated by reference in its entirety). Human islet diameters were selected from Weibull distribution (Buchwald et al., “Quantitative Assessment of Islet Cell Products: Estimating the Accuracy of the Existing Protocol and Accounting for Islet Size Distribution,” Cell Transplant 18:1223-1235 (2009), which is hereby incorporated by reference in its entirety), with the probability density function given by:

$$f ( d ) = \frac{α}{β} {( \frac{d}{β} )}^{α - 1} exp ( - {( \frac{d}{β} )}^{α - 1} ) \qquad ( 6 )$$

where α=1.5 and β=105. In specified cases (FIGS. 4K, 4L), islets were instead all generated with d=150 m.

Steady state pO₂ profiles were obtained by solving the diffusion-reaction mass balance equation (Equation 3). Solubility and diffusivity in the islets were given by αₒ_{₂}_{,islets}=7.3×10⁻⁶ mol/(m³ Pa) and Dₒ_{₂}_{,islets}=2.0×10⁻⁹ m²/s, respectively (White et al., “Perfluorocarbons Enhance Oxygen Transport in Alginate-Based Hydrogels,” Polymers for Advanced Technologies 25:1242-1246 (2014); Chapman et al., “The Mathematical Theory of Non-Uniform Gases: An Account of the Kinetic Theory of Viscosity, Thermal Conduction and Diffusion in Gases,” Cambridge University Press (1990); Mehmetoglu et al., “Oxygen Diffusivity in Calcium Alginate Gel Beads Containing Gluconobacter suboxydans,” Artificial Cells, Blood Substitutes, and Biotechnology 24:91-106 (1996), which are hereby incorporated by reference in their entirety). In this model, Rₒ_{₂}_{,i} was only defined in the islet domains according to the following:

$${R}_{{O}_{2}} ( p {O}_{2} ) = \{ \begin{matrix}0 & , p {O}_{2} < 0.08 mmHg \\ \frac{{V}_{islets}}{{α}_{{O}_{2} , islets}} \cdot ( \frac{p {O}_{2}}{{p O}_{2} + {K}_{m}} ) & , p {O}_{2} \geq 0.08 mmHg\end{matrix} \qquad ( 7 )$$

where Vᵢₛₗₑₜₛ=0.0340 mol/(m³ s) represents the O₂ consumption rate in rat islets (Avgoustiniatos et al., “Measurements of the Effective Diffusion Coefficient of Oxygen in Pancreatic Islets,” Industrial & Engineering Chemistry Research 46:6157-6163 (2007), which is hereby incorporated by reference in its entirety) and Vᵢₛₗₑₜₛ=0.0134 mol/(m³ s) in human islets (Papas et al., “Human Islet Oxygen Consumption Rate and DNA Measurements Predict Diabetes Reversal in Nude Mice,” Am. J. Transplantation 7:707-713 (2007), which is hereby incorporated by reference in its entirety), respectively. Model 3 was used to predict islet oxygenation and necrosis in cylindrical constructs implanted intraperitoneally in mice (FIGS. 4G-J), whereby a constant pO₂ of 40 mmHg was implemented on all external boundaries. The effect of variable external boundary pO₂ was also explored. This model was also used to test the hypothetical impact of alternative scaffold compositions, including PLA, solid PVDF-HFP, or porous PVDF-HFP/alginate in constructs. Solubility and diffusivity coefficients (Cardoso et al., “Fluorinated Polymers as Smart Materials for Advanced Biomedical Applications,” Polymers 10(2):161 (2018); El-Hibri et al., “Gas Transport in Poly(Vinylidene Fluoride): Effects of Uniaxial Drawing and Processing Temperature,” J Applied Polymer Science 31:2533-2560 (1986), which are hereby incorporated by reference in their entirety) for the alternative scaffold materials are listed in Table 1. Model 3 was also used to predict the influence of partial fibrosis in the same constructs (FIGS. 5K, 5M), modeled by a no flux condition implemented on one half of the exterior of a cylindrical device (SONIC versus scaffold-free control, each 20.4 mm length) containing 500 IEQ rat islets to imitate a severe case of blockage by a fibrotic layer. Oxygenation of 500 IEQ rat islets in cylindrical constructs of variable lengths (6.4 mm versus 20.4 mm) and therefore cell densities was also evaluated with this model. Finally, Model 3 was used to evaluate human islet oxygenation and necrosis in cylindrical devices (SONIC versus scaffold free) at variable densities.

Model 4 simulated steady state O₂ transport in two thick cubic (6.6×6.6×6.6 mm) devices, each containing 500 IEQ rat islets (FIGS. 6A-F): a scaffold-free control device and a SONIC device. A constant pO₂ of 40 mmHg was applied to all exterior boundaries. All physics implementations of Model 4 were identical to those of Model 3, except for the dimensions, which are defined in FIG. 8.

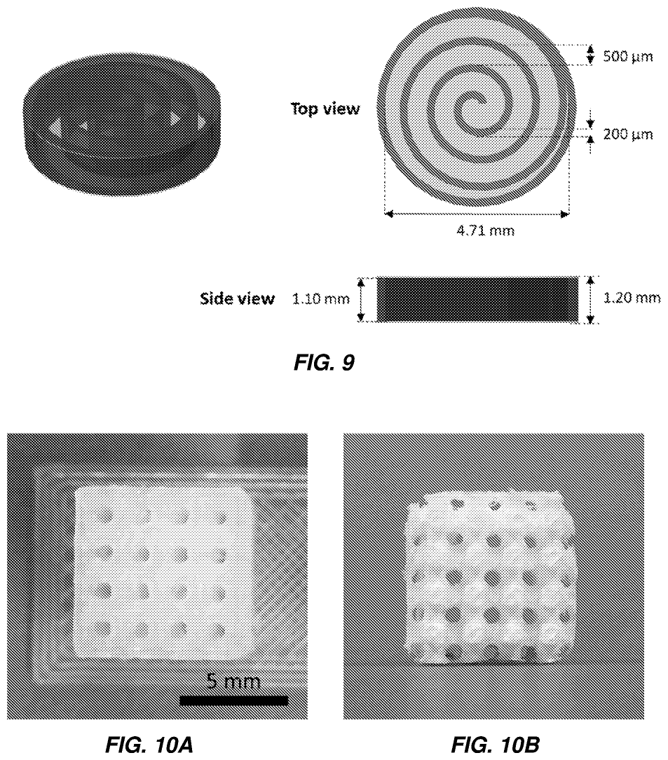

Model 5 simulated steady state O₂ transport in the SONIC spiral device and empty control containing variable loading densities of human islets. A constant pO₂ of 40 mmHg was imposed on the top and bottom boundaries whereas a no-flux condition was imposed on the lateral face, as the modeled geometry is intended to represent only the central region of a device which would be extruded radially. All other physics implementations were identical to those of Model 3, except for the dimensions, which are defined in FIG. 9.

All models were solved in COMSOL Multiphysics or COMSOL Livelink for MATLAB. In Models 3-5, all calculations were repeated for at least 3 iterations, whereby the islets were reselected and repositioned at random each time. For all calculations, a mesh was implemented using COMSOL's “Free Tetrahedral” program with the following settings: maximum element size of 100 μm, minimum element size of 1 μm, curvature factor of 0.3, resolution of narrow domains of 3.3, and maximum growth rate of 1.25. It was ensured that all results were independent of the mesh.

Statistics: All results are expressed as raw data or as mean±SD. Data from random BG measurements (FIG. 5C) were analyzed via a one-way analysis of covariance (ANCOVA) where device treatment (e.g., control device or SONIC device) was considered a discrete factor and time was considered a continuous covariate. Here, data from two-month SONIC device-treated mice (pink) were compared to data from control-treated mice (black) between days 4 and 61, while data from 6-month SONIC device-treated mice (red) were compared to data from control-treated mice (black) between days 4 and 181. Data from IPGTT studies (FIGS. 5D, 5E, and 6J) were analyzed via a two-way analysis of variance (ANOVA) where both time and treatment (e.g., diabetic control mice, healthy control mice, control device-treated mice, and SONIC device-treated mice) were considered discrete factors, followed by a Sidak's post-hocp-value adjustment for multiple comparisons. Data from the GSIS test (FIG. 5F) was analyzed via a paired two-tailed students t-test. Average population pO₂ and necrotic fraction data from modeling studies (FIGS. 4I-J) were analyzed via an unpaired two-tailed students t-test. Data from modeling studies of the thick hydrogel (FIGS. 6C-D), variable scaffold compositions, variable density studies (FIGS. 7C-D), and variable external pO₂ were analyzed via a two-way ANOVA followed by Sidak's post hoc p-value adjustment for multiple comparisons. Statistical significance was concluded at p<0.05.

### Example 1—Design and Fabrication of the SONIC Device

To develop a SONIC scaffold suitable for cell encapsulation and supporting rapid O₂ transport, two structural features, difficult to achieve simultaneously, were developed. The first feature was an internal microstructure that is bi-continuous and non-wettable to provide unobstructed microchannels for O₂ flow similar to the insect tracheae. The second feature was an external surface that is wettable to allow cell-laden hydrogel precursor to infiltrate. It was expected that a hydrophobic material for the scaffold skeleton would resist water infiltration. A two-step process was designed, forming first a superhydrophobic (i.e., low surface energy and high roughness) scaffold skeleton with continuous internal pores and then an adherent hydrophilic coating only on the external surface of the scaffold.

Accordingly, poly(vinylidene fluoride-co-hexafluoropropylene) (PVDF-HFP) (FIG. 1E) was selected as the skeleton material, which imparts hydrophobicity via the highly fluorinated —CF₃ group of the HFP unit (Shi et al., “Fabrication of Poly(vinylidene Fluoride-co-Hexafluropropylene) (PVDF-HFP) Asymmetric Microporous Hollow Fiber Membranes,” Journal of Membrane Science 305:215-225 (2007), which is hereby incorporated by reference in its entirety), while providing high solubility in polar solvents via the unique polarity of the alternating —CH₂— and —CF₂— groups of the VDF unit along the polymer chain (McKeen, “Fluoropolymers,” Permeability Properties of Plastics and Elastomers (2012), which is hereby incorporated by reference in its entirety). PVDF-HFP solution could then be subjected to an immersion precipitation process to produce the interconnected, porous and rough microarchitecture via phase separation (Wang et al., “Progress Report on Phase Separation in Polymer Solutions,” Adv. Mater. 31:e1806733 (2019); Zhang et al., “A Novel Solvent-Template Method To Manufacture Nano-Scale Porous Membranes for Vanadium Flow Battery Applications,” J. Mater. Chem. A 2:9524-9531 (2014), which are hereby incorporated by reference in their entirety). Furthermore, PVDF-HFP is resistant to hydrolytic, oxidative, and enzymatic breakdown (Grainger, “Fluorinated Biomaterials,” Biomaterials Science (2020), which is hereby incorporated by reference in its entirety), and is therefore advantageous for use in vivo.

Briefly, the SONIC scaffold was fabricated using the process illustrated in FIG. 1F and described in the materials and method above. A multilayer poly(lactic acid) (PLA) mold, complementary to the targeted ladder network geometry (FIG. 1D), was first 3D printed. Subsequently, a PVDF-HFP solution (15 wt % in acetone) was filled into the PLA mold and immersed in a water/ethanol (v/v=1/1) bath for the phase separation process yielding the internal porous microstructure containing polymer-rich regions and polymer-poor regions (i.e., the air channels). Finally, the ladder-like PVDF-HFP scaffold was isolated after selective extraction of the PLA mold using chloroform (FIG. 1G).

Structural analysis confirmed that the desired macro- and micro-architecture (FIG. 1H) of the SONIC scaffold was achieved. Nano-CT imaging illustrated an internal microporous structure with pore size of a few micrometers (FIG. 1I). Additionally, renderings of 3D reconstruction images revealed that the internal air channels were indeed continuous (FIG. 1J). Furthermore, a skeletal network analysis for the polymeric skeleton and the air channels confirmed the bi-continuous structure (FIG. 1K). These characterizations confirmed that the SONIC scaffold recapitulated the essential features of the tracheal system necessary for rapid gas-phase O₂ transport.

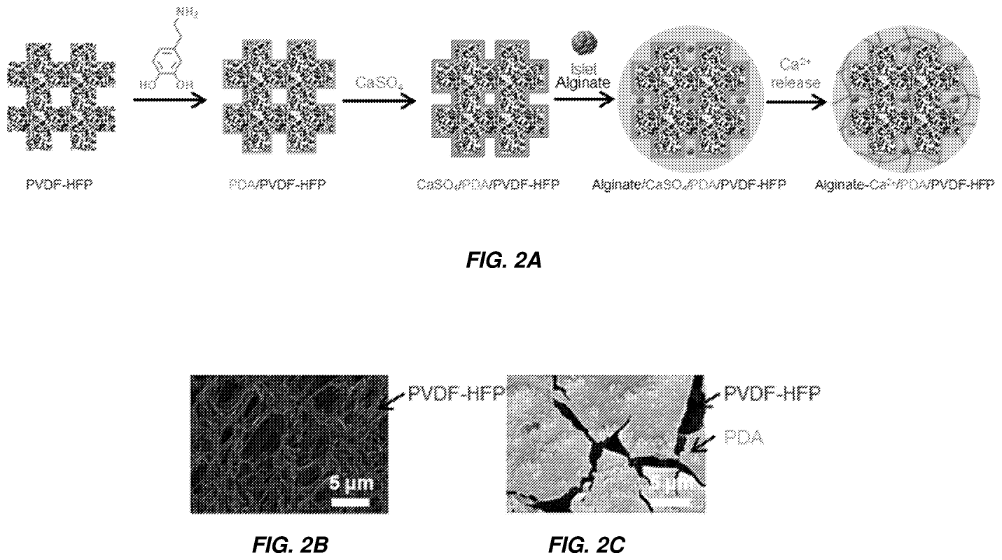

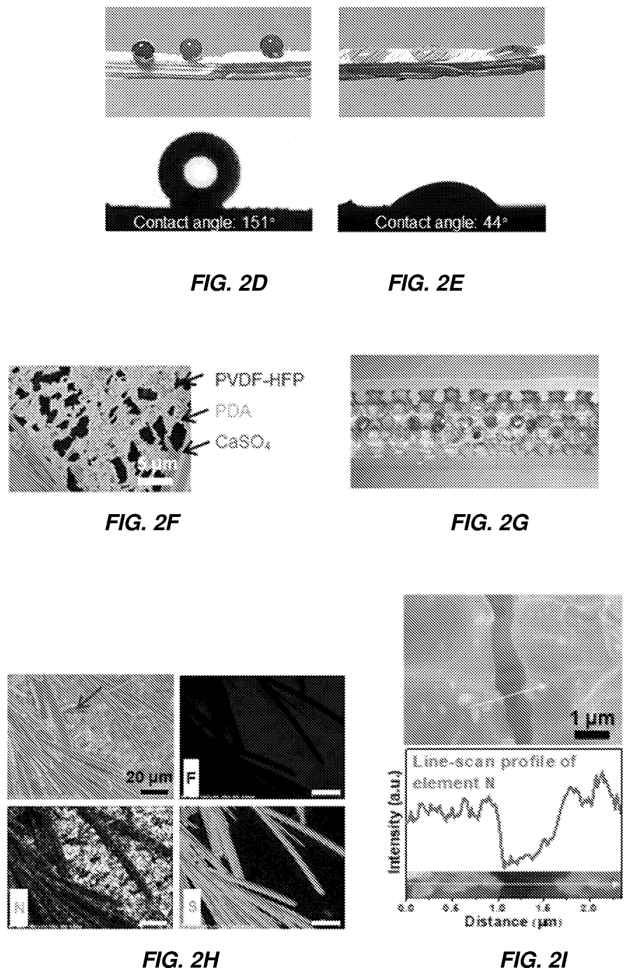

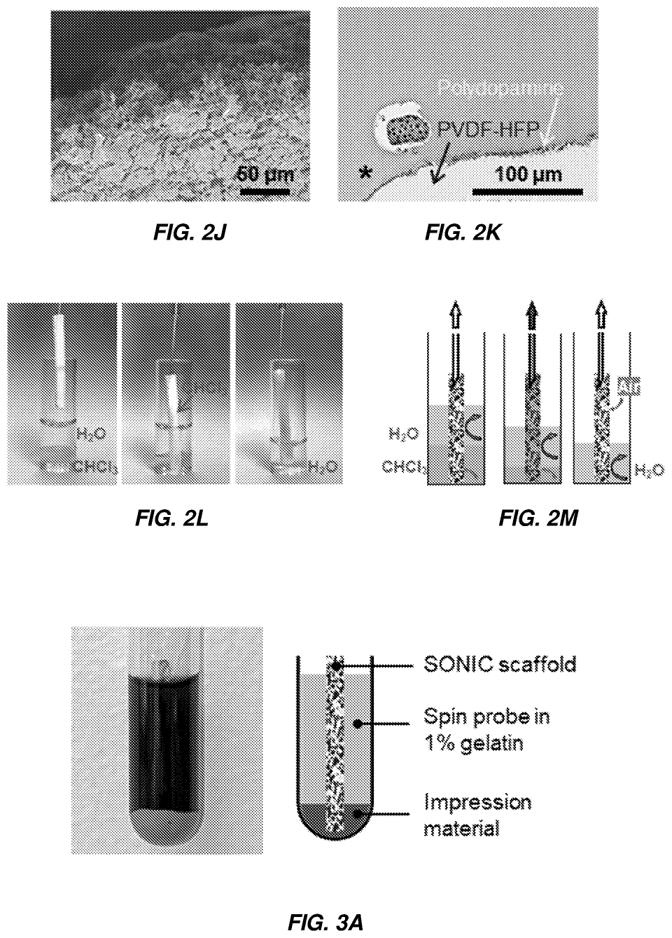

To integrate the SONIC scaffold into a hydrogel-based cell encapsulation device (FIG. 2A), the scaffold surface was first rendered wettable. Dopamine can self-polymerize to form a surface-adherent hydrophilic polydopamine film onto a wide range of substrates, including on hydrophobic surfaces (Lee et al., “Mussel-Inspired Surface Chemistry for Multifunctional Coatings,” Science 318:426-430 (2007), which is hereby incorporated by reference in its entirety). Thus, mussel-inspired hydrophilic polydopamine coating was applied on the scaffold's surface (FIGS. 2B-C) but not the internal pores. To test the efficacy of this process, water contact angle (CA) measurements were performed to characterize the wettability of the SONIC scaffold before and after polydopamine modification. The CA of polydopamine-coated SONIC scaffold decreased from the original 151° (FIG. 2D) to 440 (FIG. 2E), showing the substantially increased wettability.

Following surface hydrophilicity modification, a cell-laden hydrogel was applied via a simple in situ procedure. CaSO₄ was deposited onto the SONIC scaffold surface as a crosslinker source (FIG. 2F) for the following application of a cell-containing alginate solution (SLG 100, 2 wt %). In comparison with calcium salt (CaCl₂), which is more commonly used for alginate crosslinking, CaSO₄ has a much lower solubility (Lee et al., “Alginate: Properties and Biomedical Applications,” Progress in Polymer Science 37:106-126 (2012), which is hereby incorporated by reference in its entirety), retarding the gelation rate to provide time for the alginate solution to penetrate into the scaffold skeleton before gelling (FIG. 2G). Furthermore, the slow dissociation of Ca² from the CaSO₄ crystals produces a more uniform and mechanically stronger alginate hydrogel than the fast gelation in a CaCl₂ bath (Kuo et al., “Ionically Crosslinked Alginate Hydrogels as Scaffolds for Tissue Engineering: Part 1. Structure, Gelation Rate and Mechanical Properties,” Biomaterials 22:511-521 (2001), which is hereby incorporated by reference in its entirety).

Scanning electron microscopy (SEM)/energy dispersive X-ray spectroscopy (EDS) mapping was used to analyze the component distribution on the SONIC scaffold. Fluorine (F), nitrogen (N), and sulfur (S) were chosen as the specific elements to identify PVDF-HFP, polydopamine (PDA) and CaSO₄, respectively (FIG. 2H). The SEM/EDS line profile of N signal across a coating crack showed the lack of polydopamine inside the scaffold (FIG. 2I). In addition, an SEM cross-sectional image of the polydopamine-modified scaffold showed that the polydopamine coating was only distributed on the surface (FIG. 2J). Furthermore, hematoxylin and eosin (H&E) staining of a slice of an islet encapsulation device also confirmed that the polydopamine was only located at the interface between the scaffold and alginate hydrogel (FIG. 2K). These characterizations confirmed that hydrophilicity modification was limited to the scaffold surface and therefore unlikely to affect the hydrophobicity of internal regions of the scaffold.

A perfusion study was performed to test ability of the SONIC scaffold to prevent water from wicking into the internal pores. A cylindrical scaffold was inserted into a vial containing a water phase and a chloroform phase (FIG. 2L). The chloroform quickly penetrated the scaffold by capillary action and spread through the whole scaffold by suction force applied on the top, whereas the water was excluded and remained outside the scaffold even after all chloroform was withdrawn (FIG. 2M). The flow of chloroform throughout the scaffold also confirmed the SONIC scaffold's bicontinuous porous structure, validating visual observations in previous Nano-CT scanning characterizations (FIGS. 1J-K).

### Example 2—Rapid O₂ Transport Through the SONIC Scaffold

O₂ distribution mapping was performed on an electron paramagnetic resonance (EPR) O₂ imager to characterize O₂ transport through the SONIC scaffold (FIGS. 3A-I) (Kotecha et al., “Noninvasive Absolute Electron Paramagnetic Resonance Oxygen Imaging for the Assessment of Tissue Graft Oxygenation,” Tissue Eng. Part C Methods 24:14-19 (2018); Epel et al., “In Vivo Preclinical Cancer and Tissue Engineering Applications of Absolute Oxygen Imaging Using Pulse EPR,” J. Magn. Reson. 280:149-157 (2017), which are hereby incorporated by reference in their entirety). Briefly, a SONIC scaffold or a control PLA filament was inserted in 1% gelatin containing EPR spin probe (OX063-d24, 1 mM) in a glass tube (FIGS. 3A-C) (Kuzhelev et al., “Room-Temperature Electron Spin Relaxation of Triarylmethyl Radicals at the X-and Q-Bands,” The Journal of Physical Chemistry B 119:13630-13640 (2015), which is hereby incorporated by reference in its entirety). First, the system was deoxygenated using N₂ to reduce pO₂ close to 0 mmHg. After deoxygenation, the system was exposed to a gas mixture containing a pO₂ of 40 mmHg and the pO₂ change in gelatin was continuously monitored until a steady state (40 mmHg) was approached. The average pO₂ in gelatin with the PLA control insert slowly climbed to −35 mmHg after 11 hours (FIG. 3D). Spatial pO₂ distribution plots showed a gradient descending from top to bottom in the tangential plane and uniform profiles in the transverse plane (FIG. 3E). On the other hand, the average pO₂ in gelatin with the SONIC scaffold rapidly increased to the equilibrium level of 40 mmHg within ˜2 hours (FIG. 3F). Moreover, spatial pO₂ images in the transverse plane showed a radial gradient emanating from the location of the SONIC scaffold before achieving steady state, and profiles in the tangential plane revealed high pO₂ levels at the bottom of the sample near the SONIC scaffold, even in the first image collected after exposure to the gas mixture (FIG. 3G).

A computational model, developed to simulate spatial O₂ transfer over time in this system, was in general agreement with experimental results. In silico, the SONIC scaffold distributed O₂ more efficiently through the gelatin in comparison to the PLA control. Likewise, a clear radial O₂ gradient was observed emanating from the SONIC sample, whereas only a top-down gradient was observed in the control PLA simulation (FIG. 3H). More importantly, SONIC incorporation was predicted to equilibrate the surrounding gelatin within roughly 2 hours, whereas equilibration in the control system was significantly delayed (FIG. 3I). Mechanistically, this is because diffusive resistance to O₂ transport in the SONIC scaffold is negligible compared to that within the hydrogel, and thus a high pO₂ gradient is established between the SONIC scaffold and the hydrogel at their interface, driving O₂ transport into the hydrogel.

The EPR measurement procedure was repeated with two additional control scaffolds chosen to verify the vital role of the air channels in facilitating high oxygen permeability. One control was a commercial porous sponge comprised of hydrophilic melamine, which was selected as a simple example to confirm that a liquid-filled porous material would not provide benefit to O₂ transport. The second control was a PVDF-HFP scaffold, modified by the following two-step process to render the internal microporous channels hydrophilic: (1) ethanol was added to the tris buffer during incubation with dopamine to allow buffer solution penetration into the micropores of the scaffold, therefore enabling the application of the hydrophilic polydopamine coating within the microchannels; (2) the scaffold was treated with radio frequency plasma to further ensure its hydrophilicity. O₂ transport tests showed that these two scaffolds were significantly slower than SONIC at equilibrating the system to exposed O₂ levels. Spatial O₂ distributions of the sample containing the hydrophilic porous PVDF-HFP showed a top-to-bottom pO₂ gradient throughout the system until a steady state was achieved, rather than the radial gradient emanating from the SONIC insert (FIG. 3G), the latter of which enabled rapid and deep O₂ penetration to the bottom. The results from these two additional controls indicate the criticality of the non-wettability of the internal microchannels for enabling rapid O₂ transport in the SONIC system.

### Example 3—the SONIC Device Improves Cell Survival Under Hypoxic Conditions

In vitro tests and a complementary theoretical analysis were performed to evaluate the advantages of the SONIC scaffold for improving the viability of hydrogel encapsulated cells (FIGS. 4A-L). A computational model was developed to predict the oxygenation of INS-1 cells (2.5 million cells per mL alginate) in the control device and the SONIC device. The boundary pO₂ was set as 40 mmHg to mimic the pO₂ level in intraperitoneal cavity (Johnson et al., “Oxygen Consumption and Diffusion in Assemblages of Respiring Spheres: Performance Enhancement of a Bioartificial Pancreas,” Chemical Engineering Science 64:4470-4487 (2009), which is hereby incorporated by reference in its entirety). As expected, the control device showed large hypoxic regions in the center due to the limited O₂ transport in such a thick bulk hydrogel (FIG. 4A), with a steep pO₂ gradient decreasing from 40 mmHg at the device exterior boundary to −3 mmHg in the device center, corresponding with a drop of O₂ concentration (cₒ_{₂}) from 0.05 mM to 0.004 mM (FIG. 4B). On the other hand, all regions in the simulated SONIC device showed sufficient oxygenation throughout the entire device (FIG. 4C). The pO₂ was maintained high over 35 mmHg in the whole SONIC device with only a minor difference between central and surface regions of the cell/hydrogel phase. Correspondingly, the cell/hydrogel phase showed high cₒ_{₂} between 0.044-0.05 mM, while the SONIC scaffold showed a substantially higher equilibrium cₒ_{₂} (˜1.85 mM) due to the preferential partitioning of O₂ into the gas-containing SONIC scaffold (FIG. 4D). This suggests that the penetrating redistribution of external O₂ by the SONIC scaffold should have a significant positive impact on cell survival, especially for deeply encapsulated cells.

Results from the in vitro test were consistent with model predictions. Cell-containing devices were incubated in a hypoxic incubator with 5% CO₂ and 5% O₂ (i.e., 40 mmHg). After culturing for 48 hours, cell viability was evaluated using live/dead staining kit. Cells in the central region of the control device were dead likely due to hypoxia, with surviving cells limited to a thin region near the surface (FIG. 4E). In contrast, robust cell viability was detected throughout the SONIC device, even in the device center (FIG. 4F). These findings validated model accuracy and more importantly provided proof-of-concept for incorporating the SONIC scaffold into a device to overcome O₂ diffusion resistance to encapsulated cells.

The computational model was adapted to simulate O₂ profiles in SONIC devices, empty control devices, and control scaffold devices containing islets. In this model, 500 IEQ of rat islets were considered as discrete spheres, with diameters (30-300 μm) selected from a distribution-simulating the natural size heterogeneity of islets—and dispersed randomly within the alginate domain. Cross sectional surface plots show deficient pO₂ levels in the center of the control device (FIG. 4G), whereas pO₂ levels throughout the SONIC scaffold were maintained uniform and high at −35 mmHg near that of the external source (i.e., 40 mmHg) to provide sufficient O₂ to the surrounding islets (FIG. 4H). Here, SONIC's advantageous O₂ delivery mechanism is illustrated: external O₂ crosses only a thin barrier of the slow-diffusivity alginate before it reaches the SONIC scaffold, where it permeates rapidly throughout the structure, achieving a scaffold pO₂ level near that of the surrounding environment (FIG. 1D). This rapid equilibration is achieved because of the high O₂ permeability of the SONIC system, which is enabled by the bicontinuous air channels rather than the PVDF-HFP material itself (Table 1).

TABLE 1
O₂ Solubility, α, Diffusivity, D, and Permeability,
(αD) in Various Potential Scaffold Materials

| | α | D | (αD) |
|---|---|---|---|
| Material | (mol/m³/Pa) | (m²/s) | (mol/m/s/Pa) |
| PLA | 4.50 × 10⁻⁵ | 1.60 × 10⁻¹² | 7.20 × 10⁻¹⁷ |
| Solid PVDF-HFP | 3.29 × 10⁻⁵ | 3.50 × 10⁻¹² | 1.15 × 10⁻¹⁶ |
| Hydrophilic porous PVDF- | 1.64 × 10⁻⁵ | 1.89 × 10⁻⁹ | 3.10 × 10⁻¹⁴ |
| HFP/alginate^{†} | | | |
| SONIC | 3.90 × 10⁻⁴ | 1.80 × 10⁻⁵ | 7.02 × 10⁻⁹ |

^{†}The coefficient for the hydrophilic porous PVDF-HFP/alginate was calculated by the composition volume fraction-weighted average of the coefficients for PVDF-HFP and alginate with the assumption that the PVDF-HFP's interior microporous channels were filled with alginate.

Quantification of the oxygenation of the islet population in devices showed that the average islet pO₂ in the SONIC device was 1.8-fold higher than that in the control device (FIG. 4I), reducing the fraction of necrotic tissue 5-fold compared to islets in the control device (FIG. 4J). Additional analysis indicated that SONIC device encapsulated islets exhibited pO₂ levels descending roughly linear with their increasing diameter, with only a few of the largest islets displaying small regions falling below pO₂ levels associated with impaired function (<4 mmHg) or necrosis (<0.08 mmHg) (Buchwald P. “FEM-Based Oxygen Consumption and Cell Viability Models for Avascular Pancreatic Islets,” Theoretical Biology and Medical Modelling 6:5 (2009); Avgoustiniatos E S., “Oxygen Diffusion Limitations in Pancreatic Islet Culture and Immunoisolation,” Thesis (2002), which are hereby incorporated by reference in their entirety). In contrast, many islets, including some small ones, were anoxic in control devices. These results indicate that significantly improving the rate of O₂ transfer throughout the system bestows substantial benefits to islet oxygenation.

The simulation was repeated, but with the adaptation that each islet was implemented as 150 μm in diameter (the standard size for definition of an islet equivalent) to isolate the effect of islet distance from the surface. In the control device, it was observed that islets near the device surface were well oxygenated, while those in the center were anoxic (FIG. 4K). On the other hand, islets at all positions in the SONIC device were reasonably well oxygenated (FIG. 4L). This shows that islet oxygenation is highly dependent on encapsulation depth in exclusively hydrogel-based systems (e.g., the control device), and that SONIC scaffold incorporation effectively decouples islet oxygenation from proximity to the external O₂ source (i.e., the tissue surrounding the device in the host site).

The in vitro study and theoretical analysis performed here confirm the benefit of SONIC scaffold to improve O₂ delivery to encapsulated cells and elucidates its mechanism of action. As previously mentioned, it is often suggested that islets must be within a few hundred micrometers to the device-host boundary to avoid debilitating O₂ diffusion resistances (Dulong et al., “A Theoretical Study of Oxygen Transfer Including Cell Necrosis for the Design of a Bioartificial Pancreas,” Biotechnol. Bioeng. 96:990-998 (2007); Iwata et al., “Design of Bioartificial Pancreases From the Standpoint of Oxygen Supply,” Artif Organs 42:E168-E185 (2018), which are hereby incorporated by reference in their entirety). It was expected that the pO₂ levels in the SONIC scaffold are near those available in the transplantation site (i.e., intraperitoneal cavity). The SONIC device was designed such that any islet is within 300 μm from the SONIC scaffold or the device-host boundary. It follows that every encapsulated islet is thus within 300 μm to a high pO₂ source, regardless of its distance to the host site tissue. This not only permits the construction of thick devices, but also should allow the incorporation of significantly higher cell densities without significant impact on cell oxygenation, especially with human islets which feature lower O₂ consumption rates (Papas et al., “Human Islet Oxygen Consumption Rate and DNA Measurements Predict Diabetes Reversal in Nude Mice,” Am. J. Transplantation 7:707-713 (2007), which is hereby incorporated by reference in its entirety), and mitigate negative outcomes should the external pO₂ environment be lower. The computational analysis described herein, and corresponding in vitro tests, suggest that the SONIC device significantly improves cell survival in hydrogel-based devices.

### Example 4—the SONIC Device Enables Long-Term Diabetes Correction in Mice

Following in vitro testing and theoretical analysis, in vivo studies were pursued to test the therapeutic capability of the SONIC device (FIGS. 5A-M). Rat islets (500 IEQ per transplant) were incorporated in the SONIC devices (FIGS. 5A-B; ˜20 mm in length and ˜4 mm in diameter) or controls (bulk hydrogels of the same dimensions but without the SONIC scaffold) and transplanted into the intraperitoneal cavity of diabetic C57BL/6 mice. While both groups achieved normoglycemia within a few days after transplantation, the BG levels in the control group gradually rose and reverted to hyperglycemia after couple weeks (FIG. 5C). H&E staining of the retrieved control sample showed the islets were necrosed with severe karyorrhexis or complete loss of nuclei. It is believed that the progressive failure of control devices was mainly due to initial hypoxia and necrosis of islets in the center of device, resulting in release of danger-associated molecular patterns (DAMPs), and subsequent damage to peripheral islets from DAMP-induced immune activation (de Vos et al., “Polymers in Cell Encapsulation from an Enveloped Cell Perspective,” Adv. Drug Deliv. Rev. 67-68:15-34 (2014), which is hereby incorporated by reference in its entirety). In contrast, the BG readings in the SONIC group were under control for over 6 months (FIG. 5C). BG return to hyperglycemia after device retrieval confirmed that the function of the device was responsible for diabetes correction.

Intraperitoneal glucose tolerance tests (IPGTT) were conducted on day 60 (FIG. 5D) and day 180 (FIG. 5E) before retrievals to evaluate the kinetics of insulin release from the devices. The SONIC group showed a well-preserved glycemic profile similar to that of healthy mice: the BG returned to normoglycemia within 2 hours, indicating reasonable glucose clearance by the SONIC devices. By contrast, a poor glycemic profile was observed in the control group, as the BG remained at a high level similar to that of the diabetic control mice. A static glucose-stimulated insulin secretion (GSIS) test of the retrieved SONIC devices also showed glucose responsiveness of the encapsulated islets (FIG. 5F). In addition, the live/dead staining reveled robust islet viability (FIG. 5G). H&E staining and insulin/glucagon immunostaining further confirmed the viability and function of islets in the retrieved devices. Both the peripheral islets (FIG. 5H) and central islets (FIG. 5I) in the SONIC device showed intact morphology and positive insulin and glucagon expression.

A transverse section of a retrieved device at 6 months confirmed that both the islets close to surface and deep within the structure were viable (FIG. 5J). This was consistent with a selected transverse surface plot from the computational model, which suggested that islets in both central and peripheral regions would be highly oxygenated (FIG. 5K).

The foreign-body reaction (FBR) is a ubiquitous phenomenon for almost all foreign implants, which induces fibrosis deposition on the implant surface, occasionally leading to a complete coverage by the formation of a collagenous fibrotic capsule (Grainger D W., “All Charged up About Implanted Biomaterials,” Nat. Biotechnol. 31:507-509 (2013); Welch et al., “Antifibrotic Strategies for Medical Devices,” Adv. Drug Deliv. Rev. 167:109-120 (2020), which are hereby incorporated by reference in their entirety). Deposited fibrosis restricts the mass transfer at the implant/host interface and affects the viability of the encapsulated cells. Alginate is one of the most widely used materials for cell encapsulation due to its good biocompatibility. In addition, it has been reported that alginate capsules or fibers larger than 1.5 mm in diameter have a significantly mitigated fibrotic response (Watanabe et al., “Millimeter-Thick Xenoislet-Laden Fibers as Retrievable Transplants Mitigate Foreign Body Reactions for Long-Term Glycemic Control in Diabetic Mice,” Biomaterials 255:120162 (2020); Vlahos et al., “Muted Fibrosis from Protected Islets,” Nature Biomedical Engineering 2:791-792 (2018); Veiseh et al., “Size- and Shape-Dependent Foreign Body Immune Response to Materials Implanted in Rodents and Non-Human Primates,” Nat. Mater. 14:643-651 (2015), which are hereby incorporated by reference in their entirety). The diameter of this SONIC device is around 4 mm which is suggested to mitigate the fibrotic response. In acellular studies, minimal cellular overgrowth was observed on the retrieved device surface. For the islet-containing devices, most regions of the retrieved devices remained free of fibrosis (FIGS. 5I-J), though a mild cellular deposition was observed on partial regions (FIGS. 5H and 5L). Nonetheless, the islets near regions with deposited fibrosis remained healthy and no necrosis was observed (FIG. 5L). The computational model was adapted to simulate the effect of fibrosis coverage, implemented by setting a no-flux boundary condition on one half of the device surface, simulating an extreme case where fibrosis is assumed to eliminate O₂ transport entirely. While the simulated fibrotic side of the control device was hypoxic, with islets exhibiting high levels of necrosis, the simulated islets in the SONIC device were adequately oxygenated at both the fibrotic side and the unblocked side (FIG. 5M). Near homogeneous pO₂ levels within the scaffold illustrate that the SONIC scaffold “carries” O₂ from high pO₂ regions to low pO₂ regions over millimeter scale distances, effectively redistributing the available O₂ throughout the device.

### Example 5—the SONIC System Enables Diabetes Correction in Mice with Thick Device

Successful in vivo results of the ˜4 mm diameter device motivated the exploration of yet a thicker device design (FIGS. 6A-M). The computational model was adapted to simulate the effects of increasing device size prior to fabrication and in vivo testing. A cubic (6.6×6.6×6.6 mm) control device and SONIC device were considered, each including 500 IEQ of size-distributed rat islets (FIGS. 6A-B). Remarkably, in silico islets in the 6.6 mm-thick SONIC device were similarly oxygenated (with similarly negligible necrosis levels) as the former 4.2 mm device due to the rapid O₂ redistribution by the gas-phase transport through the SONIC scaffold (FIGS. 6C-D). The simulation, repeated but with all islets being 150 μm in diameter, showed that the pO₂ of control islets decreased with distance from the external boundary (FIG. 6E), whereas even the innermost islets in the SONIC device exhibited high pO₂ levels (FIG. 6F). This indicates that the advantage of SONIC device (decoupling islet oxygenation from distance to the host site) was maintained even at this thicker scale.

A 4-layer scaffold was then fabricated using a new printed mold to test the model predictions (FIG. 10). A 6.6 mm-thick SONIC device was prepared using the same procedure as described earlier (FIG. 2A). Rat islets (500 IEQ per transplant) were incorporated in devices and transplanted into the intraperitoneal cavity of diabetic C57BL/6 mice (n=10), with 5 retrieved at day 60 and the remainder retrieved at day 120 (FIGS. 6G-H). Strikingly, all mice achieved normoglycemia within a few days after transplantation, with 4 out of the 5 mice in the long-term study maintaining normoglycemia over 4 months (FIG. 6I). Additionally, the BG returned to hyperglycemia after device retrieval, confirming the role of the device in diabetes correction. An IPGTT performed on day 58 showed that the devices restored normoglycemia within 120 min, though the profile in device-treated mice showed a statistically significant delay in comparison to healthy control mice (FIG. 6J). BG monitoring was extended for an additional 1 hour to monitor potential hypoglycemia; 6.6 mm SONIC treated mice showed a slight overcorrection to −70 mg/dL at 150 min (compared to −120 mg/dL in healthy control mice) but stabilized near this value at the 180 min time point. The observed response delay and hypoglycemic overcorrection is likely because of the significant diffusion distance for insulin secreted from deeply encapsulated islets. Nonetheless, subsequent live/dead staining further confirmed islet viability (FIG. 6K). Finally, H&E staining and insulin/glucagon immunostaining further demonstrated maintained viability and function of islets in the retrieved devices (FIGS. 6L-M). This collective analysis indicates that the SONIC device was able to maintain islet survival and function in constructs significantly larger than traditional structures.

### Discussion of Examples 1-5

The preceding Examples describe the design and testing of an insect-inspired scaffold (SONIC) which features internal continuous air channels for rapid O₂ delivery to hydrogel encapsulated cells. Incorporation of this scaffold into bulk hydrogels containing islets (the SONIC device) supported high islet viability and robust function even in devices with a thickness of 6.6 mm. The SONIC scaffold was comprised of a hydrophobic polymer, the fluoropolymer PVDF-HFP, and the internal continuous air channels were created by a phase separation process. A hydrophilic polydopamine coating was applied to the external scaffold surface to provide a compatible interface between the hydrophobic SONIC scaffold and hydrophilic hydrogel, while maintaining internal hydrophobicity to avoid water penetration into the air channels. The latter feature was verified as critical for enabling the high O₂ permeability of the scaffold. Finally, the alginate hydrogel phase was structurally interlocked with the skeleton of the SONIC scaffold to prevent hydrogel detachment during retrieval of the transplanted device.

O₂ supply is a primary limiting factor for cell encapsulation, especially for cells with high O₂ consumption rates such as islets. To overcome this limitation, much attention has been deservedly focused on exogeneous O₂ supply, such as O₂-generating (Pedraza et al., “Preventing Hypoxia-Induced Cell Death in Beta Cells and Islets via Hydrolytically Activated, Oxygen-Generating Biomaterials,” Proc. Natl. Acad. Sci. USA 109:4245-4250 (2012); Wang et al., “An Inverse-Breathing Encapsulation System for Cell Delivery,” Science Advances 7:eabd5835 (2021); C A Patent Application No. 2924681 to Tempelman et al.; Wu et al., “In Situ Electrochemical Oxygen Generation with an Immunoisolation Device,” Annals of the New York Academy of Sciences 875:105-125 (1999), which are hereby incorporated by reference in their entirety) and O₂-filling devices (Ludwig et al., “Favorable Outcome of Experimental Islet Xenotransplantation Without Immunosuppression in a Nonhuman Primate Model of Diabetes,” Proc. Natl. Acad. Sci. USA 114:11745-11750 (2017); Evron et al., “Long-Term Viability and Function of Transplanted Islets Macroencapsulated at High Density are Achieved by Enhanced Oxygen Supply,” Sci. Rep. 8:6508 (2018), which are hereby incorporated by reference in their entirety). However, O₂ transport within the hydrogel-encapsulated cell component is driven by gradients of O₂ tension and remains slow due to the poor O₂ permeability in aqueous media. Few efforts have focused on improving O₂ transport within the cell encapsulation domain itself. Some reports have explored the effect of perfluorocarbon (PFC) emulsion incorporation in hydrogels due to the high O₂ solubility (Goh et al., “Limited Beneficial Effects of Perfluorocarbon Emulsions on Encapsulated Cells in Culture: Experimental and Modeling Studies,” J Biotechnol. 150:232-239 (2010); White et al., “Perfluorocarbons Enhance Oxygen Transport in Alginate-Based Hydrogels,” Polymers for Advanced Technologies 25:1242-1246 (2014), which are hereby incorporated by reference in their entirety), and slightly higher diffusivity (O'Brien et al., “Diffusion-Coefficients of Respiratory Gases in a Perfluorocarbon Liquid,” Science 217:153-155 (1982), which is hereby incorporated by reference in its entirety) in PFC compared to hydrogels. However, these systems generally only yield modest benefits because of the limited improvement in O₂ permeability in composite systems where the phase with the higher permeability (in this case, PFC) is dispersed (Lewis A S., “Eliminating Oxygen Supply Limitations for Transplanted Microencapsulated Islets in the Treatment of Type 1 Diabetes,” Thesis (2008), which is hereby incorporated by reference in its entirety). The bicontinuous gas phase, endowed with extremely high permeability, incorporated into the hydrogel by the SONIC scaffold facilitates the rapid permeation of O₂ throughout the device, thereby enormously improving the effective O₂ permeability of the system. Regardless of design, the current SONIC system is reliant on the O₂ available in the transplantation site; however, it is believed that the SONIC scaffold could be used to further enhance O₂ supply to hydrogel encapsulated cells even in devices which provide exogeneous supply.

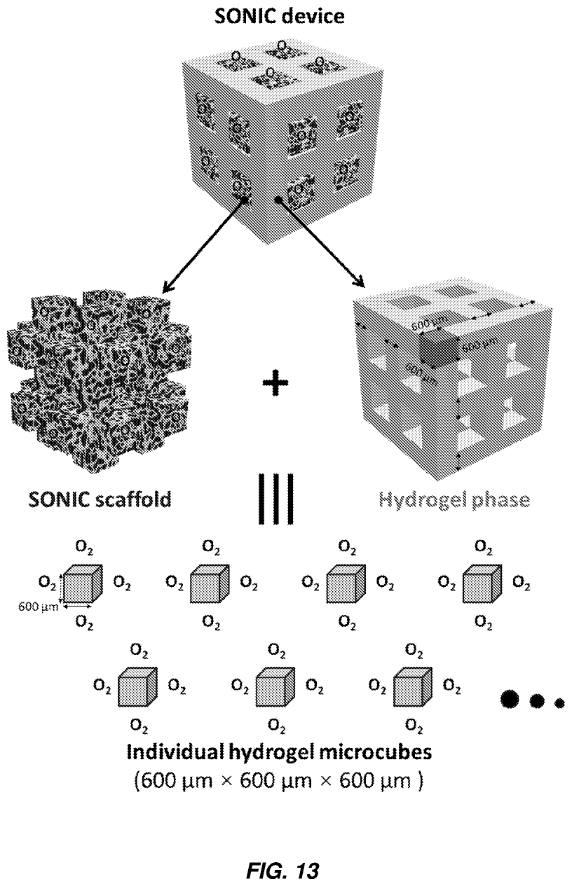

The mechanism of improved O₂ distribution via SONIC can be visualized accordingly: the continuous hydrogel phase of the device can be considered as comprising small cubic regions of ˜600×600×600 μm (FIG. 13). Each of these hypothetical “microcubes” are adjacent to the SONIC scaffold which contains uniform pO₂ levels comparable to those of the transplantation site. Thus, it is as if each hypothetical microcube itself was implanted individually in the intraperitoneal space. Furthermore, the interconnection of the hypothetical microcubes ensured that other water-phase nutrients (e.g., glucose), found in the host site in significantly higher concentrations than O₂, permeate throughout the hydrogel phase (Tannock, “Oxygen Diffusion and the Distribution of Cellular Radiosensitivity in Tumours,” British J. Radiol. 45:515-524 (1972), which is hereby incorporated by reference in its entirety).

A successful islet delivery implant must not only maintain cell survival but also ensure timely release of insulin to prevent postprandial hyperglycemia and overcorrection into hypoglycemia. The delay in IPGTT observed in mice treated with the thick SONIC device (FIG. 6J) indicate that, for islet delivery, the possibility of simply extending the device structure in all three dimensions may be limited. However, cellular treatments for hemophilia and liver diseases also utilize high cell payloads (Roth et al., “Nonviral Transfer of the Gene Encoding Coagulation Factor VIII in Patients with Severe Hemophilia A,” N. Engl. J Med. 344:1735-1742 (2001), which is hereby incorporated by reference in its entirety) without the requirement of phasic therapeutic release. Therefore, the thick SONIC system may find broader utility in these applications.

3D printing of the mold used to form the SONIC scaffold enables the scaffold to be fabricated in scaled-up dimensions (e.g., in multiple layers or extended in length to tens of centimeters) in a wide range of designs (e.g., toroidal, spiral). See, e.g., FIGS. 7A-F, 10B, 11, 12. An advantageous configuration of an islet delivery device is a planar hydrogel disk (1.2 mm thickness) incorporating an internal SONIC scaffold configured in an Archimedean spiral with a 500 μm gap between turns (FIGS. 7A-F). The small thickness of 1.2 mm obviates limitations associated with delays in insulin release, and the spiral structure ensures that each islet is within 250 μm of a high pO₂ source in the SONIC scaffold (FIG. 7A). The capacity of this construct to accommodate variable densities (4-10%) of human islets was assessed in comparison to a control construct without the SONIC scaffold using the computational model (FIG. 7B). Model predictions indicate significantly higher oxygenation of islets in the SONIC spiral device relative to the control construct, and negligible necrosis levels up to 8% (v/v) human islet loading density (FIGS. 7C-F). If extended radially, this construct could support a curative islet dose of 500 k IEQ within a disk approximately 11 cm in diameter.

In summary, this invention provides a solution to the poor transport of O₂ in traditionally employed bulk hydrogels of cell encapsulation systems. SONIC's mimicry of the insect tracheal system yields a cell encapsulation device that is amenable to increased cell density, fibrotic blockage, and—most notably—substantially increased device thickness without sacrificing cell oxygenation, in effect, decoupling cell survival from its distance to the external supply. Ultimately, these advantages imparted by the SONIC scaffold represent a promising platform for translatable encapsulation devices requiring high cell payloads.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An implantable cell containing device comprising:
a scaffold and
a cell-containing hydrogel encapsulating the scaffold,
wherein the scaffold comprises a tracheal-like internal system of continuous air-filled, hydrophobic micro-channels that traverse the scaffold's dimensions, and a hydrophilic external surface.

2. The implantable cell containing device of claim 1, wherein the scaffold comprises a polymeric material.

3. The implantable cell containing device of claim 1 or claim 2, wherein the scaffold comprises a hydrophobic material.

4. The implantable cell containing device of claim 1, wherein the scaffold comprises a fluorinated polymer material.

5. The implantable cell containing device of claim 4, wherein the fluorinated polymer material is selected from poly(vinylidene fluoride-co-hexafluoropropylene) (PVDF-HFP), poly(vinylidene fluoride) (PVDF), polyvinylidene difluoride, polytetrafluoroethylene (PTFE), poly(vinylidene fluoride-co-trifluoroethylene) (P(VDF-TrFE)), poly(vinylidene fluoride-co-tetrafluoroethylene) (P(VDF-TFE)), poly(vinylidene fluoride-co-chlorotrifluoroethylene) (P(VDF-CTFE)), Teflon AF® family: copolymers made from 2,2-bistrifluoromethyl-4,5-difluoro-1,3-dioxole and tetrafluoroethylene, and polychlorotrifluoroethylene (PCTFE).

6. The implantable cell containing device of claim 2, wherein the scaffold comprises a non-fluorinated polymer material chemically modified with fluoroalkysilanes.

7. The implantable cell device of claim 1, wherein the scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(1-lactide-co-ε-caprolactone), and combinations thereof.

8. The implantable cell containing device of claim 1, wherein the scaffold is a carbon material.

9. The implantable cell containing device of claim 8, wherein the carbon material is selected from the group consisting of activated carbon, carbon microbelts, graphite, carbon nanoparticles, carbon soot, carbon nanofibers, graphene and carbon nanotubes.

10. The implantable cell containing device of any one of claims 1-9, wherein the air-filled micro-channels of the scaffold comprise a hydrophobic surface.

11. The implantable cell containing device of any one of claims 1-9, wherein the microchannel surfaces of the scaffold are modified to comprise SiO₂ nanoparticles, hydrophobic attapulgite, ZnO nanorods, and combinations thereof.

12. The implantable cell containing device of any one of claims 1-11, wherein hydrophilic external surface of the scaffold comprises a hydrophilic polymer coating.

13. The implantable cell containing device of claim 12, wherein the hydrophilic polymer coating comprises a polydopamine coating.

14. The implantable cell containing device claim 12, wherein the hydrophilic polymer coating comprises a silane coating.

15. The implantable cell containing device of claim 14, wherein the silane coating comprises PEGylated silanes.

16. The implantable cell containing device of claim 1, wherein the hydrophilic external surface of the scaffold is a plasma-treated hydrophilic surface.

17. The implantable cell containing device of any one of claims 1-16, wherein the hydrophilic external surface of the scaffold and the cell-containing hydrogel are cross-linked.

18. The implantable cell containing device of any one of claims 1-17, wherein the scaffold comprises a ladder-like geometry, a spiral geometry, a toroidal geometry, planar geometry, rod-shaped geometry, tubular geometry.

19. The implantable cell containing device of any one of claims 1-18, wherein the hydrogel comprises a natural polymeric material, a synthetic polymeric material, or a combination thereof.

20. The implantable cell containing device of claim 19, wherein the hydrogel comprises a natural polymeric material selected from the group consisting of collagen, hyaluronate, fibrin, alginate, agarose, chitosan, bacterial cellulose, elastin, keratin, derivatives thereof, and combinations thereof.

21. The implantable cell containing device of claim 20, wherein the hydrogel material comprises a pure alginate, a modified alginate, or a mixture of pure and modified alginate.

22. The implantable cell containing device of claim 21, wherein the modified alginate is a zwitterionically modified alginate.

23. The implantable cell containing device of claim 19, wherein the hydrogel comprises a synthetic polymeric material selected from polyethylene glycol (PEG), poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof.

24. The implantable cell containing device of any one of claims 1-23, wherein the cells of the hydrogel comprise a preparation of single cells or a preparation of cell aggregates.

25. The implantable cell containing device of any one of claims 1-24, wherein the cells of the hydrogel comprise a preparation of primary cells or a preparation of immortalized cells.

26. The implantable cell containing device of any one of claims 1-25, wherein the cells of the hydrogel comprise a preparation of mammalian cells.

27. The implantable cell containing device of claim 26, wherein cells of the hydrogel comprise a preparation of mammalian cells selected from the group consisting of primate cells, rodent cells, canine cells, feline cells, equine cells, bovine cells, and porcine cells.

28. The implantable cell containing device of claim 27, wherein the cells of the hydrogel comprise human cells.

29. The implantable cell containing device of any one of claims 1-28, wherein the cells of the hydrogel comprise a preparation of stem cells or stem cell derived cells.

30. The implantable cell containing device of claim 29, wherein the stem cells are pluripotent, multipotent, oligopotent, or unipotent stem cells.

31. The implantable cell containing device of claim 29, wherein the preparation of stem cells is selected from the group consisting of embryonic stem cells, epiblast cells, primitive ectoderm cells, primordial germ cells, and induced pluripotent stem cells.

32. The implantable cell containing device of any one of claims 1-28, wherein the cells of the hydrogel are selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells.

33. The implantable cell containing device of claim 32, wherein the cells of the hydrogel comprise a preparation of islet cells that release insulin and glucagon.

34. The implantable cell containing device of claim 32, wherein the preparation of islet cells is a preparation of human cells, porcine cells, or rodent islets.

35. The implantable cell containing device of claim 33 or 34, wherein the preparation of islets comprise a density between 1×10³ to 6×10⁵ islet equivalents (IEQs)/mL.

36. The implantable cell delivery device of any one of claims 1-35, wherein the cell-containing hydrogel further comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

37. A method of delivering a therapeutic agent to a subject in need thereof, said method comprising:
implanting the implantable cell containing device according to any one of claims 1-36 into the subject.

38. A method of treating diabetes in a subject, said method comprising:
implanting the implantable cell containing device according to any one of claims 1-36 into the subject having diabetes.

39. The method of claim 38, wherein the cell-containing hydrogel of the device comprises a preparation of cells that release insulin, glucagon, or a combination thereof for the treatment of diabetes in the subject.

40. The method of claim 38 or claim 39, wherein the preparation of cells is a preparation of islets.

41. The method of claim 40, where the preparation of islets is a preparation of primate islets, rodent islets, canine islets, feline islets, equine islets, bovine islets, or porcine islets.

42. The method of claim 40, wherein the preparation of islets is derived from a preparation of stem cells.

43. The method of claim 42, wherein the preparation of stem cells is selected from the group consisting of embryonic stem cells, epiblast cells, primitive ectoderm cells, primordial germ cells, and induced pluripotent stem cells.

44. A method of treating a bleeding disorder in a subject, said method comprising:
implanting the implantable cell containing device of any one of claims 1-36 into the subject having a bleeding disorder.

45. The method of claim 44, wherein the bleeding disorder is selected from the group consisting of hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

46. The method of claim 44 or claim 45, wherein the cell-containing hydrogel of the device comprises a preparation of cells that release one or more blood clotting factors selected from the group consisting of Factor I, Factor II, Factor V, Factor VII, Factor VIII, Factor IX, Factor X, Factor XI, Factor XII, and Factor XIII for treatment of the bleeding disorder.

47. The method of claim 46, wherein the preparation of cells comprises recombinant myoblasts, mesenchymal stromal cells, induced pluripotent stem cell derived endothelial cells, or a combination thereof.

48. A method of treating a lysosomal storage disease in a subject, said method comprising:
implanting the implantable cell containing device of any one of claims 1-36 into the subject having the lysosomal storage disease.

49. The method of claim 48, wherein the cell-containing hydrogel of the device comprises a preparation of cells that release an enzyme selected from the group consisting of α-L-iduronidase, Iduronate-2-sulfatase, α-glucuronidase, Arylsulfatase A, alpha-Galactosidase A, and combinations thereof, for treating the lysosomal storage disease in the subject.

50. The method of claim 49, wherein the preparation of cells comprises hematopoietic stem cells, fibroblasts, myoblasts, Baby Hamster Kidney (BHK) cells, Chinese Hamster Ovary cells, Human Amniotic Epithelial (HAE) cells, or combinations thereof.

51. A method of treating a neurological disorder in a subject, said method comprising:
implanting the implantable cell containing device of any one of claims 1-36 into the subject having the neurological disorder.

52. The method of claim 51, wherein the neurological disorder is selected from the group consisting of Parkinson's disorder, Alzheimer's disease, epilepsy, Huntington's disease, Amyotrophic lateral sclerosis, chronic pain, visual loss, hearing loss, peripheral nerve injury, and spinal cord injury.

53. The method of claim 51 or claim 52, wherein the cell-containing hydrogel of the device comprises a preparation of cells that release a molecule selected from cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof, for treating the neurological disorder.

54. The method of claim 53, wherein the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, myogenic cells, embryonic stem cell-derived neural progenitor cells, or combinations thereof.

55. A method of treating a cancer in a subject, said method comprising:
implanting the implantable cell containing device of any one of claims 1-36 into the subject having cancer.

56. The method of claim 55, wherein the cell-containing hydrogel of the device comprises a preparation of cells that release a molecule selected from IL-2, endostatin, cytochrome P450 enzyme, tumor antigens, a cytokine, and combinations thereof, for treating cancer in the subject.

57. The method of claim 56, wherein the preparation of cells comprises IL-2-secreting myoblasts, endostatin-secreting cells, Chinese Hamster Ovary cells, and cytochrome P450 enzyme overexpressed feline kidney epithelial cells, irradiated tumor cells, or combinations thereof.

58. A method of treating a chronic eye disease in a subject, said method comprising:
implanting the implantable cell containing device of any one of claims 1-36 into the subject having a chronic eye disease.

59. The method of claim 58, wherein the chronic eye disease is selected from the group consisting of age-related macular degeneration, diabetic retinopathy, retinitis pigmentosa, glaucoma, macular telangiectasia, and combinations thereof.

60. The method of claim 58 or claim 59, wherein the cell-containing hydrogel of the device comprises a preparation of cells that release a molecule selected from ciliary neurotrophic factor, antagonists against vascular endothelial growth factor and platelet-derived growth factor, and combinations thereof, for treating the chronic eye disease.

61. The method of claim 60, wherein the preparation of cells comprises human retinal pigment epithelium cells, recombinant human retinal pigment epithelium cells, or a combination thereof.

62. A method of treating a kidney failure in a subject, said method comprising:
implanting the implantable cell containing device of any one of claims 1-36 into the subject having a kidney failure.

63. The method of claim 62, wherein the cell-containing hydrogel of the device comprises a preparation of cells that release a therapeutic molecule suitable for treating the kidney failure.

64. The method of claim 62 or 63, wherein the cell containing hydrogel comprises a preparation of renal proximal tubule cells, mesenchymal stem cells, and a combination thereof.

65. A method of treating a chronic pain in a subject, said method comprising:
implanting the implantable cell containing device of any one of claims 1-36 into the subject having a chronic pain.

66. The method of claim 65, wherein the chronic pain is chronic pain caused by degenerative back and knee, neuropathic back and knee, or cancer.

67. The method of claim 65 or claim 66, wherein the cell-containing hydrogel of the device comprises a preparation of cells that release a molecule selected from the group consisting of catecholamine, opioid peptides, enkephalins, and combinations thereof.

68. The method of claim 67, wherein the preparation of cell comprises chromaffin cells, neural precursor cells, mesenchymal stem cells, astrocytes, and genetically engineered cells, or a combination thereof.

69. The method according to any one of claims 37-68, wherein said implanting is carried out via a laparoscopic procedure.

70. The method according to any one of claims 37-68, wherein said implantable cell containing device is implanted intraperitoneally, percutaneously, or subcutaneously.

71. The method according to any one of claims 37-68, wherein said method further comprises:
retrieving the implantable cell containing device from the subject.

72. The method according to claim 71, wherein said method further comprises:
implanting a replacement implantable cell containing device after said retrieving.

73. A method of preparing a scaffold for a cell encapsulation system, the method comprising:
providing a mold;
introducing a polymer solution into the mold and allowing the polymer to solidify and form a three-dimensional structure comprising a network of hydrophobic microchannels throughout the three-dimensional structure; and
removing the mold to release the three-dimensional structure, thereby forming the scaffold.

74. The method according to claim 73, wherein said providing comprises 3D printing the mold.

75. The method according to claim 73, wherein the polymer solution comprises an acetone solvent, and said allowing comprises immersing the filled mold into a water/ethanol bath and/or a water bath to promote solidification.

76. The method according to claim 73, wherein said removing comprises soaking the mold and three-dimensional structure in a solvent that dissolves the mold.

77. The method according to claim 73, further comprising treating an external surface of the three-dimensional structure to render it hydrophilic.

78. The method according to claim 77, wherein said treating comprises exposing the external surface of the three-dimensional structure to plasma or a surface reactive agent that is hydrophilic.

79. The method according to claim 77, wherein the method further comprises drying the three-dimensional scaffold prior to said treating, sterilizing the three-dimensional scaffold after said treating, or both.

80. The method according to claim 73, wherein the scaffold comprises a ladder-like geometry, a spiral geometry, a toroidal geometry, planar geometry, rod-shaped geometry, tubular geometry.

81. A scaffold for use in a cell encapsulation system, the scaffold comprising:
a three-dimensional structure comprising a network of hydrophobic microchannels throughout the three-dimensional structure; and
a hydrophilic external surface of the three-dimensional structure.

82. The scaffold of claim 81, wherein the three-dimensional structure comprises a polymeric material.

83. The scaffold of claim 82, wherein the polymeric material is a fluorinated polymer material.

84. The scaffold of claim 83, wherein the fluorinated polymer material is selected from poly(vinylidene fluoride-co-hexafluoropropylene) (PVDF-HFP), poly(vinylidene fluoride) (PVDF), polyvinylidene difluoride, polytetrafluoroethylene (PTFE), poly(vinylidene fluoride-co-trifluoroethylene) (P(VDF-TrFE)), poly(vinylidene fluoride-co-tetrafluoroethylene) (P(VDF-TFE)), poly(vinylidene fluoride-co-chlorotrifluoroethylene) (P(VDF-CTFE)), Teflon AF® family: copolymers made from 2,2-bistrifluoromethyl-4,5-difluoro-1,3-dioxole and tetrafluoroethylene, and polychlorotrifluoroethylene (PCTFE).

85. The scaffold of claim 82, wherein the polymeric material is a non-fluorinated polymer material chemically modified with fluoroalkysilanes.

86. The scaffold of claim 81, wherein the three-dimensional structure comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(ε-caprolactone), poly(lactic-co-glycolic acid), poly(1-lactide-co-ε-caprolactone), and combinations thereof.

87. The scaffold of claim 81, wherein the three-dimensional structure comprises a carbon material.

88. The scaffold of claim 88, wherein the carbon material is selected from the group consisting of activated carbon, carbon microbelts, graphite, carbon nanoparticles, carbon soot, carbon nanofibers, graphene and carbon nanotubes.

89. The scaffold of any one of claims 81 to 88, wherein the microchannel surfaces of the three-dimensional structure comprise SiO₂ nanoparticles, hydrophobic attapulgite, ZnO nanorods, or combinations thereof, to render the microchannels hydrophobic.

90. The scaffold of any one of claims 81 to 89, wherein hydrophilic external surface comprises a hydrophilic polymer coating.

91. The scaffold of claim 90, wherein the hydrophilic polymer coating comprises a polydopamine coating.

92. The scaffold of claim 90, wherein the hydrophilic polymer coating comprises a silane coating.

93. The scaffold of claim 92, wherein the silane coating comprises PEGylated silanes.

94. The scaffold of any one of claims 81 to 89, wherein hydrophilic external surface is plasma-treated.

95. The scaffold of any one of claims 81 to 94, wherein the three-dimensional structure comprises a ladder-like geometry, a spiral geometry, a toroidal geometry, planar geometry, rod-shaped geometry, or a tubular geometry.

96. A method of forming a cell encapsulation system suitable for implant, the method comprising:
providing a hydrogel precursor solution comprising one or more cells suspended in the hydrogel precursor solution; and
combining the hydrogel precursor solution with a scaffold according to any one of claims 81 to 95 in a contained state, and allowing the hydrogel precursors to cross-link to form the hydrogel with the scaffold and the one or more cells embedded within the hydrogel.

97. The method of claim 96, wherein said combining is carried out in a vessel.

98. The method of claim 96, further comprising:
depositing calcium salts onto an exterior surface of the scaffold prior to said combining.

99. The method of claim 96, wherein the hydrogel comprises a natural polymeric material, a synthetic polymeric material, or a combination thereof.

100. The method of claim 99, wherein the hydrogel comprises a natural polymeric material selected from the group consisting of collagen, hyaluronate, fibrin, alginate, agarose, chitosan, bacterial cellulose, elastin, keratin, derivatives thereof, and combinations thereof.

101. The method of claim 100, wherein the hydrogel material comprises a pure alginate, a modified alginate, or a mixture of pure and modified alginate.

102. The method of claim 101, wherein the modified alginate is a zwitterionically modified alginate.

103. The method of claim 99, wherein the hydrogel comprises a synthetic polymeric material selected from polyethylene glycol (PEG), poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof.

104. The method of claim 99, wherein the one or more cells comprise a preparation of single cells or a preparation of cell aggregates.

105. The method of claim 99, wherein the one or more cells comprise a preparation of primary cells or a preparation of immortalized cells.

106. The method of claim 99, wherein the one or more cells comprise a preparation of mammalian cells.

107. The method of claim 106, wherein the mammalian cells are selected from the group consisting of primate cells, rodent cells, canine cells, feline cells, equine cells, bovine cells, and porcine cells.

108. The method of claim 106, wherein the mammalian cells are human cells.

109. The method of claim 99, wherein the one or more cells comprise a preparation of stem cells or stem cell derived cells.

110. The method of claim 109, wherein the stem cells are pluripotent, multipotent, oligopotent, or unipotent stem cells.

111. The method of claim 109, wherein the preparation of stem cells is selected from the group consisting of embryonic stem cells, epiblast cells, primitive ectoderm cells, primordial germ cells, and induced pluripotent stem cells.

112. The method of claim 99, wherein the one or more cells are selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells.

113. The method of claim 99, wherein the one or more cells comprise a preparation of islet cells that release insulin and glucagon.

114. The method of claim 113, wherein the preparation of islet cells is a preparation of human islets, porcine islets, or rodent islets.

115. The method of claim 113, wherein the preparation of islets comprise a density between 1×10³ to 6×10⁵ islet equivalents (IEQs)/mL.

116. The method of claim 99, wherein the hydrogel precursor solution further comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.
